(19) United States
(12) Patent Application Publication
(10) Pub. No.: US 2025/0236644 A1
(43) Pub. Date: Jul. 24, 2025

(54) De Novo Designed Stimulus-responsive Two-state Hinge Proteins

(71) Applicant: University of Washington, Seattle, WA (US)

(72) Inventors: David Baker, Seattle, WA (US); Florian Praetorius, Seattle, WA (US); Philip Leung, Seattle, WA (US); Maxx Tessmer, Seattle, WA (US); Adam Broerman, Seattle, WA (US); Cullen William Demakis, Seattle, WA (US); Acacia Frances Dishman, Seattle, WA (US); Arvind Pillai, Seattle, WA (US); Abbas Idris, Seattle, WA (US)

(21) Appl. No.: 19/023,949

(22) Filed: Jan. 16, 2025

Related U.S. Application Data
(60) Provisional application No. 63/623,417, filed on Jan. 22, 2024.

(51) Int. Cl. C07K 14/00

(52) U.S. Cl. CPC C07K 14/001; C07K 2319/01

(57) ABSTRACT

Polypeptides having an amino acid sequence at least 75% identical to the amino acid sequence of SEQ ID NO:1-20 and 33-58 are provided, fusion proteins thereof, kits thereof, and methods for their use as sensing devices and for other uses.

### FEDERAL FUNDING STATEMENT

This invention was made with government support under Grant No. HR0011-21-2-0012, awarded by the Defense Advanced Research Projects Agency. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jan. 15, 2025 having the file name “24-2155-US.xml” and is 62,987 bytes in size.

### BACKGROUND

The generation of proteins that can switch between two quite different structural states is a difficult challenge for computational protein design, which usually aims to optimize a single, very stable conformation to be the global minimum of the folding energy landscape. Design of such proteins requires reframing the design paradigm towards optimizing for more than one minimum on the energy landscape, while simultaneously avoiding undesired off-target minima.

### SUMMARY

In a first aspect, the disclosure provides polypeptides comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-20, wherein (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted. In one embodiment, the polypeptide comprises the amino acid sequence selected from SEQ ID NO:1-20.

In another embodiment, the polypeptide comprises the amino acid sequence selected from SEQ ID NO:1-3. In another embodiment, the disclosure provides fusion proteins, comprising (a) a polypeptide of any embodiment of the first aspect; and (b) one or more functional domains fused to the N-terminus and/or C-terminus of the polypeptide. In one embodiment, the one or more functional domains are selected from the group consisting of a detectable protein, a polypeptide binding domain for a target, a protein enzyme, and an oligomerization domain. In another embodiment, the one or more functional domains comprise the amino acid sequence selected from SEQ ID NO:21-28, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted. In a further embodiment, the fusion protein comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:29-32, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted.

In a second aspect, the disclosure provides polypeptides comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:33-43 and 49-63; wherein (i) any N-terminal M residue is optional and may be present or may be deleted, and (ii) any C-terminal GSGSHHWGSTHHHHHH (SEQ ID NO:44) sequence is optional and may be present or may be deleted. In one embodiment, the polypeptide comprises the amino acid sequence selected from SEQ ID NO:33-43 and 49-63; wherein (i) any N-terminal M residue is optional and may be present or may be deleted, and (ii) any C-terminal GSGSHHWGSTHHHHHH (SEQ ID NO:44) sequence is optional and may be present or may be deleted.

In other aspects, the disclosure provides nucleic acids encoding the polypeptide or fusion protein of any embodiment or combination of embodiments of the first and second aspect of the disclosure; expression vectors comprising such nucleic acids operatively linked to a promoter sequence; and host cells comprising such polypeptides, fusion proteins, nucleic acids, and expression vectors. The disclosure also provides kits comprising (a) one or more polypeptide of any embodiment of the first aspect of the disclosure; and (b) one or more polypeptide comprising any embodiment of the second aspect of the disclosure.

### DESCRIPTION OF THE FIGURES

FIG. 1. Strategy for designing proteins that can switch between different conformations. A) Left: reaction scheme for a protein that undergoes a conformational change and can bind an effector in one (circle) but not in the other conformational state (square). Right: Energy landscape for the system shown on the left. B) Schematic representation of the hinge design approach. Alpha-helices are represented as circles (top view, top) or cylinders (side view, bottom). From left to right: A previously designed repeat protein serves as the first conformation of the hinge. To generate the second conformation a copy of the repeat protein is moved by shifted alignment along a pivot helix, causing a rotation (top and bottom, indicated by circular arrow) and a translation along the helix axis (bottom). The first 4 helices of the original protein form domain 1 of the hinge, the last 4 helices of the rotated copy form domain 2, and an additional helix is copied over from the original protein to serve as an effector peptide that can bind to this second conformation of the hinge. Both domains of the hinge are connected into one continuous chain using fragment-based loop closure, and a single amino acid sequence is designed to be compatible with both conformations. C) Design steps from B illustrated using cartoon representations of an exemplary design trajectory. D) Exemplary design models of a designed hinge protein in state X (left), state Y (center), and in state Y bound to an effector peptide (right).

FIG. 2. Experimental validation of peptide-binding hinges. A) Design models of hinges and peptides in state X (left model) and state Y bound to the peptide (right model). Shades behind models in state X and Y indicate the corresponding states Y and X, respectively. B) Fluorescence Polarization (FP) titrations with a constant concentration of TAMRA-labeled peptide (0.1 nM for cs074 and cs221; 0.5 nM for cs201; 1 nM for cs094, cs207, and js007) and varying hinge concentrations. Circles represent data points from four independent measurements, lines are fits of standard binding isotherms to all data points, dissociation constants (K_{D}) are obtained from those fits. C,D) Distance distributions between spin labels covalently attached to cysteine side chains. Solid lines are obtained from DEER experiments without or with an excess of peptide, shaded areas are 95% confidence intervals, and dashed lines are simulated based on the design models for state X or the state Y complex. For each hinge two different label site pairs were tested, one in which the distance was expected to decrease with peptide binding (C) and one in which the distance was expected to increase upon peptide binding (D). Chemically synthesized peptides were used for all measurements except for cs074 site pair 1, for which sfGFP-peptide fusion was used. For design cs094, the residual state X peak in presence of the peptide can be explained by incomplete binding either due to weak binding affinity or to insufficient peptide concentration.

FIG. 3. Close agreement between crystal structures and design models for both designed states. A) Design model of hinge cs207 in state X overlaid with crystal structure of hinge cs207 crystallized without peptide. Right panel shows a close-up view of the side chains in the interface between the two hinge domains. B) Design model of the cs207 state Y hinge-peptide complex overlaid with crystal structure of hinge cs207 co-crystallized with peptide cs207B. Right panel shows a close-up view of the side chains in the interface between hinge and peptide. C) Design model of hinge cs074 in state Y overlaid with crystal structure of hinge cs074 co-crystallized with peptide cs207B. Representative electron densities for all crystal structures are shown in FIG. 24. RMSD values between design model and experimental structure are given in Table 8. D) Left: Components for design of a C3-symmetric homotrimer with three cs221 hinge arms. Center: Design model of the hinge-armed trimer in state X (top) and in state Y (bottom). Right: nsEM class averages of the trimer in absence of peptide (top) and in presence (bottom) of peptide cs221B.

FIG. 4. Quantitative analysis of conformational changes in designed hinge proteins A) FRET-based characterization of three extended hinges. From left to right: cylindrical representation of extended hinges and their corresponding target peptides (cs201B, cs221B, cs074B) with stars indicating attachment sites for fluorescent dyes; fluorescence spectra (excitation at 520 nm) of labeled hinge without or with target peptide; FRET-based binding titrations (excitation 520 nm, emission 665 nm) at 2 nM labeled hinge and varying peptide concentrations fitted with standard binding isotherms (solid lines); time course after mixing 2 nM (cs201F, cs074F) or 5 nM (cs221F) labeled hinge and 100 nM peptide fitted with a single-exponential equation (black line); apparent rate constants obtained from single-exponential kinetic fits plotted against absolute peptide concentrations (circles) and fitted with a linear equation (black line). Dotted lines in spectra indicate acceptor and donor emission peaks. B) Kinetic model describing the coupling of the conformational equilibrium to the binding equilibrium. X and Y: hinge in state X and Y, respectively; P: peptide; YP: peptide bound to hinge in state Y. k₁, k₋₁, k₋₂, and k₋₂ are the microscopic rate constants. C) FP characterization of unlabeled extended hinge cs074F. From left to right: binding titration at 0.1 nM TAMRA-labeled peptide and varying hinge concentrations; time course after mixing 2 nM TAMRA-labeled peptide and 100 nM hinge fitted with a single-exponential equation (black line); apparent rate constants obtained from single-exponential kinetic fits plotted against absolute hinge concentrations (circles) and fitted with a linear equation (black line). D) FRET-based reversibility experiment using the labeled extended hinge cs201F introduced in C). Hinge concentration is 30 nM for all traces; 1 μM peptide is added at t=0, 3 μM unlabeled competitor hinge is added after 1 h. E) Top from left to right: schematic representation of the inpainting procedure that adds two helices to the peptide cs074B yielding a three-helix bundle (3hb); cylindrical representation of 3hb_05 bound to hinge cs074; overlay of design model and crystal structure of 3hb_05. Bottom from left to right: SEC traces for hinge cs074, 3hb_05, and a mixture of both; FRET-based titration of 2 nM extended labeled hinge cs074F and varying concentrations of 3hb_05 fitted with a standard binding isotherm (back line); Distance distributions obtained from DEER experiments as described in FIG. 2 (cs074, cs074+peptide cs074B, cs074+3hb_05).

FIG. 5. Controlling the conformational pre-equilibrium affects peptide binding. A) Left: Schematic representation of a hinge containing two cysteine residues that can form a disulfide bond in state X but not in state Y, effectively locking the hinge in state X under oxidizing conditions. Upon addition of reducing agent DTT the disulfide bond is broken and the conformational equilibrium is restored. Right: FP-based titration of 1 nM TAMRA-labeled peptide and a hinge with state X disulfide or the parent hinge without cysteines under oxidizing or reducing conditions. B) From left to right: schematic representation of a hinge that is disulfide-locked in state Y; time course after mixing 2 nM TAMRA-labeled peptide and 50 nM locked hinge (red) or original hinge without cysteines fitted with a single-exponential equation (black line); apparent rate constants obtained from single-exponential kinetic fits plotted against absolute hinge concentrations (circles) and fitted with a linear equation (black line). C) Tuning the pre-equilibrium with point mutations. Top left: Cartoon representation of hinge cs221 highlighting positions of point mutations. Top right: Dissociation constants (K_{D}) and observed binding rate constants (kₒₙ). Bottom left: FP-based titration of 0.1 nM or 1 nM TAMRA-labeled peptide cs221B and varying concentrations of hinge variants containing one or two point mutations. Bottom center: Apparent rate constants obtained from single-exponential kinetic fits plotted against absolute hinge concentrations (circles) and fitted with a linear equation (black line). Bottom right: DEER distance distribution for the double mutant cs221-V111L-A114T in absence of peptide (gray) in comparison to the original cs221 with and without peptide. Vertical lines serve as guide to the eye indicating state X and state Y distances.

FIG. 6. Diversity of hinge structures and conformational changes. Light points represent individual designs, with positions as a function the angle change of the hinge (state Y-state X, N-terminus-midpoint-C-terminus angle), measured in degrees, and the change in radius of gyration of the hinge (state Y-state X) in angstrom as computed by PyRosetta™. Black points are representative examples and are depicted as cartoon models.

FIG. 7. SEC Characterization of additional hinges not shown in FIG. 2. Purification runs of hinges (left) and sfGFP-peptide fusions (center) were performed on Superdex™ 75 Increase 10/300 GL columns (Cytiva) except for traces with label “S200” that were run on a Superdex™ 200 Increase 10/300 GL column. SEC binding experiments were performed on Superdex™ 200 Increase 10/300 GL columns (Cytiva).

FIG. 8. Size exclusion chromatography (SEC) of the hinges shown in FIG. 2. Purification runs of hinges (left) and sfGFP-peptide fusions (center) were performed on Superdex™ 75 Increase 10/300 GL columns (Cytiva). SEC binding experiments were performed on Superdex™ 200 Increase 10/300 GL columns (Cytiva).

FIG. 9. Additional characterization experiments. A) FP-based titration using TAMRA-labeled peptide at 1 nM and varying concentrations of hinge. For cs217 the signal never reached a plateau, thus only a lower bound for the K_{D} can be estimated. B) FP-based kinetics experiment using 5 nM TAMRA-labeled peptide cs269B and varying concentrations of hinge cs269 as indicated by plot labels. Top: All kinetic traces were fitted using a single-exponential equation (black lines). Bottom: Apparent rate constants (points) from the single exponential fits plotted against the hinge concentration and fitted as linear (black lines). The slope of the linear fit gives the observed on rate kₒₙ. C) DEER distance distributions for hinges cs129 (one site pair) and cs217 (two site pairs) in absence of peptide and with excess peptide. Dashed lines are simulated distributions based on design models, solid lines are fits to the experimental data, shaded areas are confidence intervals of these fits. D) DEER control experiment using DHR82 (the parent of hinge cs074) labeled at the same sites as cs074. Top: DHR82 shows a sharp peak that does not shift upon addition of the peptide cs074B. Bottom: The distance distribution for cs074 that is shown in FIG. 2 is shown again as comparison to the DHR82 distribution. The hinge cs074 in absence of peptide shows a slightly broader peak than the parent DHR, suggesting increased flexibility and conformational breathing.

FIG. 10. Redesign of hinges to bind other target peptides. An experimentally tested hinge (top, cs201) is redesigned using a one-sided two-state design approach to bind to different target peptides. Hinges csw13 and csw20 are designed to bind to peptides cs221B and cs074B, respectively, while having a backbone conformation that is similar to the parent cs201 that binds cs201B. Left: design models, right: FP titrations using 1 nM peptide (cs201B, cs221B, cs074B, js007B) and varying concentrations of hinge (from top to bottom: cs201, csw13, csw20). csw13 is an example of a successful orthogonal redesign that specifically binds the new target peptide cs221B while not binding the parent target peptide cs201B or the off-target peptides cs074B or js007B. csw20 is a less orthogonal example that binds the new target peptide cs074B most strongly but still binds to the parent-target peptide cs201B and to the off-target peptide cs221B albeit weaker than the new target.

FIG. 11. Circular Dichroism (CD) thermal melts of the six hinge proteins shown in FIG. 2. A) CD spectra at 25° C., at 95° C., and at 25° C. after cooling back from 95° C. B) Mean Residue Ellipticity (MRE) at 222 nm during temperature ramping from 25° C. to 95° C. Gray lines indicate MRE of 0.

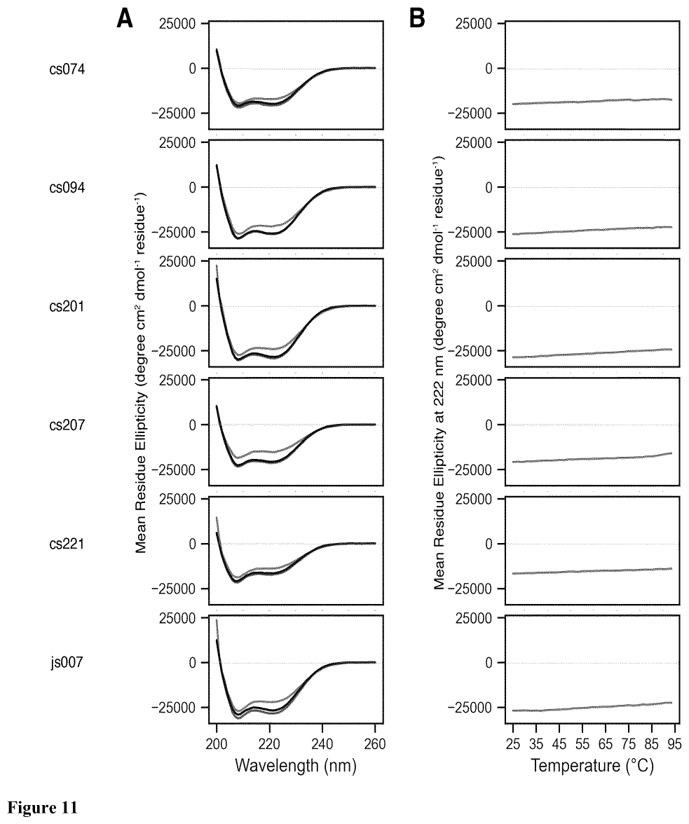

FIG. 12. negative stain electron microscopy on hinge-armed trimers. A,B) Field-of-view electron micrographs of hinge-armed trimers in absence (A) or presence (B) of peptide cs221B. C,D) Class averages from one round of classification (20 classes) using particles obtained from hinge-armed trimers in absence (C) or presence (D) of peptide cs221B.

FIG. 13. Additional FRET experiments. A) Fluorescence spectra of proteins (2 nM) without or with peptide cs074B (5 nM for cs074 variants, 2 μM for DHR82). Proteins from top to bottom: cs074F labeled with AlexaFluor™ 555 (=donor only), cs074F labeled with AlexaFluor™ 647 (=acceptor only), cs074F labeled with a 1:1 mixture of AlexaFluor™ 555 and AlexaFluor™ 647 (=donor+acceptor), DHR82 labeled with a 1:1 mixture of both dyes. DHR82 shows no significant change in FRET upon addition of the peptide. B) Titrations of the same proteins as in A at 1.2 nM and peptide cs074B at varying concentrations. Left: Acceptor emission upon donor excitation, right: donor emission upon donor excitation. C) Replicate titrations of the FRET-labeled extended hinges shown in FIG. 4 (2 nM hinge), and FP titration of the unlabeled extended hinge cs221F (right, 1 nM TAMRA-peptide cs221B).

FIG. 14. Full kinetics measurements of the extended hinges shown in FIG. 4. Columns 1-3: FRET kinetics using extended hinges labeled with AlexaFluor™ 555 and AlexaFluor™ 647 at a constant concentration (2 nM for cs201F and cs074F, 5 nM for cs221F) and corresponding peptides at varying concentrations. Column 4: FP kinetics using TAMRA-labeled peptide cs074B at 2 nM and extended hinge cs074F at varying concentrations. All kinetic traces (rows 1-8) were fitted using a single-exponential equation (black lines). Row 9 shows apparent rate constants from the single exponential fits plotted against the hinge concentration and fitted as linear (black lines). The slope of the linear fit gives the observed on rate kₒₙ.

FIG. 15. FP kinetics measurements of hinges shown in FIG. 2. Rows 1-8: TAMRA-labeled peptide at a constant concentration (2 nM for cs074, 50 nM for cs207, 5 nM for cs201, cs221, and js007) was mixed with hinge at varying concentrations (labels in each plot indicate the hinge concentration for the corresponding experiment). All kinetic traces were fitted using a single-exponential equation (black lines). Row 9: Apparent rate constants from the single exponential fits plotted against the hinge concentration and fitted as linear (black lines). The slope of the linear fit gives the observed on rate kₒₙ.

FIG. 16. Additional three-helix bundles that bind cs074. A) SEC binding experiments. Left: Overlaid chromatograms of three-helix bundles, hinge cs074 and mixtures of both show clear binding. Right: Overlaid chromatograms of three-helix bundles, parent DHR82 and mixtures of both show no significant binding. B) FRET-based titration experiments using 2 nM labeled cs074F and varying concentrations of 3hb or peptide cs074B show that the 3hb designs bind to the target hinge with nanomolar affinities and cause a conformational change. C) DEER experiments with MTSL-labeled cs074 show that the 3hb designs cause the same conformational change as the original peptide cs074B. Shaded areas indicate 95% confidence intervals.

FIG. 17. Additional three-helix bundles. A) Models of hinges in state X and in state Y bound to a three-helix bundle (3hb). B) SEC binding experiments of hinge, 3hb, and mixture of both show clear monodisperse complex peaks. C) SEC binding experiments of the same hinges with the original peptides fused to superfolder green fluorescent protein (sfGFP). For cs230, cs245, and cs269 the hinge-peptide complex shows higher-order peaks while the corresponding hinge-3hb peaks look much cleaner. For cs244, the original peptide showed no clear binding in the SEC experiment, while 3hb28 shows clear binding.

FIG. 18. FRET-based quantitative analysis of the interaction between cs221F and 3hb21. Individual kinetic traces were obtained using a constant hinge concentration of 5 nM and varying 3hb concentrations as indicated by plot labels. Single exponential fits give apparent rate constants that increase linearly with the total 3hb concentration (points in pseudo-first order plot). The linear fit of kₐₚₚ against 3hb concentration gives an observed on rate that is 5 times faster than the observed on rate of the original peptide. FRET titration of 2 nM hinge cs221F and varying concentrations of 3hb gives a KD below 2 nM which is at least 20 times stronger than the KD of the original peptide.

FIG. 19. Structural validation of three-helix bundles. A,B) Overlay of design model and crystal structure in side view (left) and top view (right) for designs 3hb05 (A, also shown in FIG. 4E) and 3hb12 (B).

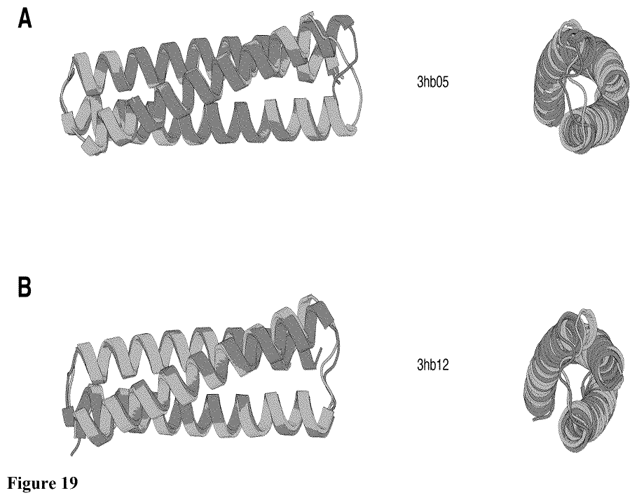

FIG. 20. Additional FP data on disulfide variants and point mutants. A) FP titration of the cs221 locked Y hinge variant shown in FIG. 5B in comparison to the original cs221 hinge. B) Full FP kinetics experiment for the cs221 locked Y hinge variant shown in FIG. 5B. Individual kinetic traces were obtained using 1 nM TAMRA-labeled peptide cs221B and varying hinge concentrations as indicated by plot labels. Single exponential fits give apparent rate constants that increase linearly with the total hinge concentration (points in pseudo-first order plot). C) Full-range individual plots of the FP titrations shown in FIG. 5C. D) FP titrations and kinetics for the MTSL-labeled variants of hinge cs221-V111L-A114T. The spin labels have no measurable effect on affinity or association kinetics.

FIG. 21. Point mutations in hinge cs221. A) Renderings of cs221 variants in state X (left) and state Y bound to the peptide (right). B) Schematic energy landscapes illustrating the effect of exemplary mutations. Left: Mutation A114T shifts the pre-equilibrium towards state Y, thus increasing kₒₙ and lowering K_{D}. Right: Mutation L66T does not affect the pre-equilibrium and, in turn, has no effect on kₒₙ. The effect on K_{D} can be explained by an allosteric destabilization of the state Y-peptide complex. C) Kinetic model (left) and relevant equations (right).

FIG. 22. Full FP kinetics experiments for the cs221 mutants shown in FIG. 5C. Rows 1-8: TAMRA-labeled peptide cs221B at a constant concentration of 5 nM was mixed with hinge at varying concentrations (labels in each plot indicate the hinge concentration for the corresponding experiment). All kinetic traces were fitted using a single-exponential equation (black lines). Row 9: Apparent rate constants from the single exponential fits plotted against the hinge concentration and fitted as linear (black lines). The slope of the linear fit gives the observed on rate kₒₙ.

FIG. 23. DEER data on hinge variants that populate both states in absence of peptide. Distance distributions obtained from DEER experiments with the original hinge cs221 in absence of peptide and in presence of peptide as well as of the double mutant cs221-V111L-A114T in absence of peptide (top, mutant Apo,) and in presence of peptide (bottom, mutant Holo).

FIG. 24. Representative electron densities (2mFo-DFc, 1σ) of crystal structures shown in FIG. 3. A) cs207A (see FIG. 3A). B) cs207+cs207B (see FIG. 3B). C) cs074+cs074B (see FIG. 3C). Two different views of the same density are shown for each structure (top and bottom).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), RosettaCommons.org, and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be deleted).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application

In a first aspect, the disclosure provides polypeptides comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-20,
wherein (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted.

The polypeptides of this aspect are “hinge” proteins that populate one designed state in the absence of ligand and a second designed state in the presence of ligand. The sequences of SEQ ID NO:1-20 are provided in Table 1.

TABLE 1

| SEQ | |
|---|---|
| ID NO | Name/sequence |
| | hinge cs074 (minimal) |
| 1 | NPDNEEAVKTAVRLARELLKVAEELKERAEKTGDPRLLLLAAEAIAWAIEAVFLA |
| | AKASENTEGALEAARAAVKLAEVAKRIAKLLQRDAKKEGDPELLKLALRALELAV |
| | RAVELAIKENPDN |
| | hinge cs201 (minimal) |
| 2 | AIKRNPDNEEAIKTALRLARELRKVAKELIERARKTGDAELLKKALEAARVAVEA |
| | VRLAAEYNKENAEKMAELLVELAELAREVADVLIELAEKTGDPELLKKALEVLEE |
| | AVEAVRLAIEYDPDH |
| | hinge cs221 (minimal) |
| 3 | SDDEEVKEVVKKALEAALKSKDEEVIRLLLLAAVLAAAAARSGSPEEKLEIAKKA |
| | LELAMKSKDEEVIRLALLAAVLAARSDDEEVLK |
| | hinge cs074 |
| 4 | MSGDEEVQEAVERAEELREEAEELIKKARKTGDPELLRKALEALKEAVRAVKEAI |
| | KRNPDNEEAVKTAVRLARELLKVAEELKERAEKTGDPRLLLLAAEAIAWAIEAVF |
| | LAAKASENTEGALEAARAAVKLAEVAKRIAKLLQRDAKKEGDPELLKLALRALEL |
| | AVRAVELAIKENPDNEEAVETAKRLAEELRKVAELLEERAKETGDPELQELAKRA |
| | KEVADRARELAKKSNPNNGSGSHHWGSTHHHHHH |
| | hinge cs094 |
| 5 | MSGDEEERLRQEVEKAEKELEKLAKQSTDEEVRKMVREVAKQLRRLAEEAIRQKD |
| | DEALRQATEVVKMVQEAVKVAQKTTDQKVILLLLAVALVAIKVAQLAIRSDDTEA |
| | LRLARIVIRAVQLLVKLVQKTTDPEVRRTALRVAELLAKLAKEAIERNDEEALRE |
| | ASEVVKEVQELVKEAEKSTDEEEIRELLQRAEERIREAQERIREGSGSHHWGSTH |
| | HHHHH |
| | hinge cs129 |
| 6 | HHHHHHSGGSSDEEEARELIERAKEAAERAQEAAERTGDPRVRELARELKRLAQE |
| | AAEEVKRDPSSSDVNKALKLIVELIEQAVRILQAAEEVGDPELRELARELVRLLV |
| | EMAEQVQRNPSSEDEVRALKLVVQAIEAAIRAAHAAQRTGDPEVDKLAKELVRLA |
| | VEAAEEVMRNPSSEEVNEALKKIVKAIQEAVESLREAEESGDPEKREKARERVRE |
| | AVERAEEVQRDPSS |
| | hinge cs201 |
| 7 | MSGDEEVQEAVERAEELREEAEELIKKARKTGDPELLRKALEALKEAVRAVREAI |
| | KRNPDNEEAIKTALRLARELRKVAKELIERARKTGDAELLKKALEAARVAVEAVR |
| | LAAEYNKENAEKMAELLVELAELAREVADVLIELAEKTGDPELLKKALEVLEEAV |
| | EAVRLAIEYDPDHEEAVETAKRLAEELRKVAELLEERAKETGDPELQELAKRAKE |
| | VADRARELAKKSNGSGSHHWGSTHHHHHH |
| | hinge cs207 |
| 8 | MSGTEDERRELEKVARKAIEAAREGNTDEVREQLQRALEIARESGSEEAFKLALE |
| | VVRRVAEVAARAGNVEAVKEALRVALEIVKEAMELIKDPEAIVRLALEAVRVVAE |
| | VAARAGAVEAVKVALRVALEIAKIAGTEEAVRLALEVVKRVSDIAKKAGNEDAVK |
| | EAEEVRKKIEEESGGSGSHHWGSTHHHHHH |
| | hinge cs217 |
| 9 | MSGEMKEEIRRLAEELEKKTKDEEVKELARKAAELAEKSDNEEVLEVVKEALRAA |
| | LKSKDEEVRRLLLEAAVLAAAAESSGSPEEKLEVALRAIRLAEKSKDEEVRRLAL |
| | EAAVLAAESDDEEVLEEVLRALRRAEESKDEEERREELREAVRRAREGSGSHHWG |
| | STHHHHHH |
| | hinge cs221 |
| 10 | MSGEMKEEIRRLAEELRERTKDEEVRELAREAARLAEESDDEEVKEVVKKALEAA |
| | LKSKDEEVIRLLLLAAVLAAAAARSGSPEEKLEIAKKALELAMKSKDEEVIRLAL |
| | LAAVLAARSDDEEVLKKVKEALEKAMESKDVEEIRERLREAVEVARAGSGSHHWG |
| | STHHHHHH |
| | hinge js007 |
| 11 | MSGSEEVNERVKQLAEKAKEATDKEEVIEIVKELAELAKQSTDPNLVAEVVRALT |
| | EVAKTSTDTELIQEIVKVLLELARRLTDPQLLLEVLKSIAELLKELAEKTGNETA |
| | KLAALVAQIAAEVVEMALRAEKTHPGSEIVKLAVELVQKVAEIVLIAAQLMLDKP |
| | NSDEVRKVLKEVEKVAREALKALREAKRHPDSQKARDEIKEASQKAEEVKERIER |
| | AQEGSGSHHWGSTHHHHHH |
| | hinge cs230 |
| 12 | MSGTKEEKERIERIEKEVRSPDPENILEAVRKALELFRENPSEEAKELLKRAIEA |
| | ALRSEDERAILEALRAALEAYRQGAGDREGLLEAIRVAIRRAAESEDERAILEAV |
| | RAARILLEAERTEEAKELLRVAIERAKKSPDPEAQREAKRAEEELRKEDGSGSHH |
| | WGSTHHHHHH |
| | hinge cs253 |
| 13 | MSGKEKAIKKLEEAIKLAEKVGSEEAKILKEIAEALIHAVKGDAEEAEEKARRAS |
| | EEAKKVGSELAKILKLLAEALIHAVLAQKGDADAKEALKKAAELLKKAAEKVGSE |
| | EAKILKLIAEAVIAAVEGDKEKAIELLERAVELAKKVGSEEAKILKEIAEALIEA |
| | VKGGSGSHHWGSTHHHHHH |
| | hinge cs269 |
| 14 | MSGDEERVRELAERASREAERAVRSGSEESLEEALRNVEELIRKSDSEEARVRVA |
| | EAAVRAAAEAAARSGTVEAAAVAVIFTARVLQIVSSSEEAVVRVLAAAAAAAARV |
| | AVELGTEEALETALRFVELLVRLADSDEARQRVLRSASESARRSAEESGSERARE |
| | VAERFRERIERNQGSGSHHWGSTHHHHHH |
| | hinge cs287 |
| 15 | MSGDEELRQLLREAEELVREAREELKENPDDRETAEKAVRKAREALDRAREALKE |
| | LPTEEAIRRLARIAKEAVEVAKRAVEAVPESERVLLEAARVALEAVEAARRALEL |
| | AKGENTEEIEEAARILARVAKRAVEVAKRALEARPDSPELAEEAVKLAEEALEAA |
| | ERALKVLPTDEAIEILREIAERAKEIARIARESVPDSPEVKKNAQRVEEKADRSL |
| | EEAEKRNGSGSHHWGSTHHHHHH |
| | hinge cs292 |
| 16 | MSGEEEERLRRAVEEAVREAERVREEAKKSGSEEAREVAREVREAAERAREVAEE |
| | AIRSGDPRALRLAEEVLEAVRKAAEVAVEAIKSGSEEAIELAREVAEAARRAAEV |
| | AREAIRVDDPRALRIAELAAEAVEAVAKAVEAAVLAIKSGSEEAIEAARLVAEAA |
| | YRAIEVAREAIRTGDERALELAREVLRSVIEAAKIAKEAIKSGSEEEIEKAIRVA |
| | KEAYREAERAERRIREGGSGSHHWGSTHHHHHH |
| | swapped target hinge csw13 |
| 17 | MSGNEEVKKLVEEAKKYLKEAEEYIKKAKKTGNEELLEKALKLLEKALELVKKAI |
| | ELDPNNKEAIEQAVEIARALNEVAELLIEEAKKTGNEELLEKALEALEAANEALK |
| | LAIENNENDKELLAEIAVELAEVGVLLAEVLIEEAKKTGDPELLKLALEVLEAAV |
| | EALELALKLDPNNEEAVELAEEAAKLLEEVAKELKEEAKKTGDPELEKLAKEAEE |
| | LAKRAKELAERQRGSGSHHWGSTHHHHHH |
| | swapped target hinge csw20 |
| 18 | MSGMEEVKELVEKAEELAKEAEELIKKAKKTGNPELLKEALRKLKEAVELAKEAV |
| | ELDPEHEEAIELLARLAEKLLEVAKLLIELAKKTGNPELLKEALRALKEAREALR |
| | LAAEHAPEDAKLLARLAARLAELALLIAKILIELAKKTGNPELLLKALKVLTLAV |
| | EAVEAALEADPRLKEAVRVAKELAEVLKELAKLAKELAKKTGNEELKEAAKEAEE |
| | VAKRAEELAREAEGSGSHHWGSTHHHHHH |
| | variant cs221_lockedX |
| 19 | MSGEMKEEIRRLAEELRERTKDEEVRELAREAARLAEESDDEEVKEVVKKALEAA |
| | LKSKDEEVIRLLLLAAVCAAAAARSGSPEEKLEIAKKALELAMKSKDEEVIRLAL |
| | LAAVLACRSDDEEVLKKVKEALEKAMESKDVEEIRERLREAVEVARAGSGSHHWG |
| | STHHHHHH |
| | variant cs221_lockedY |
| 20 | MSGEMKEEIRRLAEELRERTKDEEVRELAREAARLAEESDDEEVKEVVKKALEAA |
| | LKSKDEEVIRLLLCAAVLAAAAARSGSPEEKLEIAKKALELAMKSKDEEVIRLAC |
| | LAAVLAARSDDEEVLKKVKEALEKAMESKDVEEIRERLREAVEVARAGSGSHHWG |
| | STHHHHHH |

In one embodiment, the polypeptide comprises the amino acid sequence selected from SEQ ID NO: 1-20, wherein (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted. In a further embodiment, the polypeptide comprises the amino acid sequence selected from SEQ ID NO: 1-20.

In one embodiment, the polypeptide comprises the amino acid sequence selected from SEQ ID NO: 1-3, wherein (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted. In a further embodiment, the polypeptide comprises the amino acid sequence selected from SEQ ID NO:1-3. The polypeptides of SEQ ID NO:1-3 are minimal portions of certain hinge proteins that can be used, for example, in the fusion proteins disclosed herein (for example, the fusion proteins of SEQ ID NO:29-32).

In one embodiment of any aspect of the polypeptides of the disclosure, amino acid substitutions relative to the reference peptide domains are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the disclosure provides fusion proteins comprising a polypeptide of the first aspect and one or more additional functional domains added at the N-terminus and/or the C-terminus of the polypeptide. Any suitable functional domain(s) may be added as suitable for an intended purpose, including but not limited to detectable protein, a polypeptide binding domain for a target, a protein enzyme, and an oligomerization domain, etc.

In one embodiment, the one or more functional domains comprise the amino acid sequence selected from SEQ ID NO:21-28, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted. The amino acid sequences of SEQ ID NO:21-28 are provided in Table 2. These functional domains are all used in the fusion proteins disclosed herein (for example, the fusion proteins of SEQ ID NO:29-32).

TABLE 2

| SEQ | |
|---|---|
| ID NO | |
| 21 | DEEVQEAVERAEELREEAEELIKKARKTGDPELLRKALEALECAVRAVEEAIKRN |
| | PDNDEAVETAVRLARELKKVAEELQERAKKTGDPELLKLALRALKVAVRAVKLAI |
| | KS |
| 22 | DEAVETAVRLARELKKVAEELQERAKKTGDPELLKLALRALEVAVRAVELAIKSN |
| | PDNEEAVETAKRLAEELRKVAELLEERACETGDPELQELAKRAKEVADRARELAK |
| | KSNPNNGSHHHHHH |
| 23 | MSGDEEVQEAVERAEELREEAEELIKKARKTGDPELLRKALEALEEAVRAVEEAI |
| | CRNPDNDEAVETAVRLARELKKVAEELQERAKKTGDPELLKLALRALEVAVRAVE |
| | LAIKSNPDNDEAVETAVRLARELKKVAEELQERAKKTGDPELLKLALRALEVAVR |
| | AVEL |
| 24 | DEAVETAVRLARELKKVAEELQERAKKTGDPELLKLALRALEVAVRAVELAIKSN |
| | PDNEEAVETAKRLAEELRKVAELLEERAKETGDPCLQELAKRAKEVADRARELAK |
| | KSNPNNGSGSHHWGSTHHHHHH |
| 25 | MSGEMVEEIEKLAEELAKKTKDCEVKKLAKKAAELAKKSTDEMKKEIVKDALELA |
| | LRTKDEEVIRLALKAAVLAAE |
| 26 | IVKKALELAMESKDVEVIRLALEAAVLAARSTDPEKKKCVYEALEKAMESKDEEE |
| | IKKELKAAVEKARSTEGSGSHHWGSTHHHHHH |
| 27 | MSGPELVLKALENMVRAAHTLAEIARDNGNEEWLEAAARLAELVAKAAEELAREA |
| | RKEGNLELALKALQILVNAAYVLAEIARDRGNEELLEYAARLAEEAARQAAEIAI |
| | KAARKGNLELALEALRILNEAARVLARIAHHRGNQELLKKAQHLTEASAAISKML |
| | AAIAAATGGGEMIGEIAGLAAELIFRTDDKEVRELAARAAGLAAG |
| 28 | LVKKALEKAMESKNVEAIRNMLKVAVEAARSGGGRQWAVVLSIAETAGKMGVTME |
| | FHVSGNEVKVVIKGLHESQQEQLLEDVLRTAEKQGVRVRIRFKGDTVTIVVREGS |
| | GSHHWGSTHHHHHH |

The polypeptide and the one or more functional domains may be joined by an amino acid linker, or may be directly adjacent in the fusion protein with no interviewing linker. When an amino acid linker is present, it may comprise any amino acid linker suitable for an intended use.

In one embodiment, the fusion protein comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:29-32, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted. In another embodiment, the fusion protein comprises the amino acid sequence selected from SEQ ID NO:29-32, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted. In a further embodiment, the fusion protein comprises the amino acid sequence selected from SEQ ID NO:29-32. The amino acid sequences o SEQ ID NO:29-32 are provided in Table 3.

TABLE 3

| SEQ | |
|---|---|
| ID NO | AA Sequence |
| | FRET(extension) cs074F |
| 29 | DEEVQEAVERAEELREEAEELIKKARKTGDPELLRKALEALECAVRAVEEAIKRN |
| | PDNDEAVETAVRLARELKKVAEELQERAKKTGDPELLKLALRALKVAVRAVKLAI |
| | KSNPDNEEAVKTAVRLARELLKVAEELKERAEKTGDPRLLLLAAEAIAWAIEAVF |
| | LAAKASENTEGALEAARAAVKLAEVAKRIAKLLQRDAKKEGDPELLKLALRALEL |
| | AVRAVELAIKENPDNDEAVETAVRLARELKKVAEELQERAKKTGDPELLKLALRA |
| | LEVAVRAVELAIKSNPDNEEAVETAKRLAEELRKVAELLEERACETGDPELQELA |
| | KRAKEVADRARELAKKSNPNNGSHHHHHH |
| | FRET(extension) cs201F |
| 30 | MSGDEEVQEAVERAEELREEAEELIKKARKTGDPELLRKALEALEEAVRAVEEAI |
| | CRNPDNDEAVETAVRLARELKKVAEELQERAKKTGDPELLKLALRALEVAVRAVE |
| | LAIKSNPDNDEAVETAVRLARELKKVAEELQERAKKTGDPELLKLALRALEVAVR |
| | AVELAIKRNPDNEEAIKTALRLARELRKVAKELIERARKTGDAELLKKALEAARV |
| | AVEAVRLAAEYNKENAEKMAELLVELAELAREVADVLIELAEKTGDPELLKKALE |
| | VLEEAVEAVRLAIEYDPDHDEAVETAVRLARELKKVAEELQERAKKTGDPELLKL |
| | ALRALEVAVRAVELAIKSNPDNEEAVETAKRLAEELRKVAELLEERAKETGDPCL |
| | QELAKRAKEVADRARELAKKSNPNNGSGSHHWGSTHHHHHH |
| | FRET(extension) cs221F |
| 31 | MSGEMVEEIEKLAEELAKKTKDCEVKKLAKKAAELAKKSTDEMKKEIVKDALELA |
| | LRTKDEEVIRLALKAAVLAAESDDEEVKEVVKKALEAALKSKDEEVIRLLLLAAV |
| | LAAAAARSGSPEEKLEIAKKALELAMKSKDEEVIRLALLAAVLAARSDDEEVLKI |
| | VKKALELAMESKDVEVIRLALEAAVLAARSTDPEKKKCVYEALEKAMESKDEEEI |
| | KKELKAAVEKARSTEGSGSHHWGSTHHHHHH |
| | hinge-armed trimer |
| 32 | MSGPELVLKALENMVRAAHTLAEIARDNGNEEWLEAAARLAELVAKAAEELAREA |
| | RKEGNLELALKALQILVNAAYVLAEIARDRGNEELLEYAARLAEEAARQAAEIAI |
| | KAARKGNLELALEALRILNEAARVLARIAHHRGNQELLKKAQHLTEASAAISKML |
| | AAIAAATGGGEMIGEIAGLAAELIFRTDDKEVRELAARAAGLAAGSDDEEVKEVV |
| | KKALEAALKSKDEEVIRLLLLAAVLAAAAARSGSPEEKLEIAKKALELAMKSKDE |
| | EVIRLALLAAVLAARSDDEEVLKLVKKALEKAMESKNVEAIRNMLKVAVEAARSG |
| | GGRQWAVVLSIAETAGKMGVTMEFHVSGNEVKVVIKGLHESQQEQLLEDVLRTAE |
| | KQGVRVRIRFKGDTVTIVVREGSGSHHWGSTHHHHHH |

In a second aspect, the disclosure provides polypeptides comprising the amino acid sequence selected from SEQ ID NO:33-43 and 49-63; wherein (i) any N-terminal M residue is optional and may be present or may be deleted, and (ii) any C-terminal GSGSHHWGSTHHHHHH (SEQ ID NO:44) sequence is optional and may be present or may be deleted. The polypeptides of SEQ ID NO:33-43 and 49-63 are effector peptides, which provide for effector-induced switching between the two states of the hinge proteins of the first aspect. In one embodiment, the polypeptides of this second aspect comprise the amino acid sequence selected from SEQ ID NO:33-43 and 49-63. The amino acid sequences of these effector polypeptides are provided in Table 4, and the specific polypeptide or fusion protein of the first aspect of the disclosure that they bind to is also provided.

TABLE 4

| SEQ | |
|---|---|
| ID NO | AA Sequence |
| | effector protein 3hb03 (to be used with hinge cs074(SEQ |
| | ID NO: 1, 4 or 29)) |
| 33 | MSGMAAKVREEAERVREEARETRERAREAAERGEVSEETAAALLLEAAVLELKAVL |
| | LELEARRIYEEAGPEEREVAEEARRLAEEARRLAREARELAREAEEGGSGSHHWGS |
| | THHHHHH |
| | effector protein 3hb04 (to be used with hinge cs074(SEQ |
| | ID NO: 1, 4 or 29)) |
| 34 | MSGGEAEELREEAERARRRAEEARERAREALERARRGEASEEHAAALLAEAAVLEL |
| | KAVLLTLEARRLEKELGGDERAREALEAAEEARRAAREARREAEEAFEAASGSGSH |
| | HWGSTHHHHHH |
| | effector protein 3hb05 (to be used with hinge cs074 (SEQ |
| | ID NO: 1, 4 or 29)) |
| 35 | MSGEEAERLRREAERNREKAEEQREKAKKAYEKAKKGEASEEHAAALLAEAAVLEL |
| | KAVLLTLEARRLYKELGGDERAREALEAAERAREAAREAREVAEKAYDAASGSGSH |
| | HWGSTHHHHHH |
| | effector protein 3hb08 (to be used with hinge cs074 (SEQ |
| | ID NO: 1, 4 or 29)) |
| 36 | MSGGEERAREVREEAERTRREAEELRERAEEALESGEASEEHAAALLLEAAVLELK |
| | AVLLELEARRLREEHPTEEAREAYEAAERAREAAREALEAAREALEKSGSGSHHWG |
| | STHHHHHH |
| | effector protein 3hb11 (to be used with hinge cs074 (SEQ |
| | ID NO: 1, 4 or 29)) |
| 37 | MSGGKAEEARRLAEKALEAARKALEYARKAKEALEEGKSEEVVAAYLLEAAVEDLK |
| | AVLLRLEARRLAEESDEEARRETERLLEEARRAREEARRLRREIEERKGSGSHHWG |
| | STHHHHHH |
| | effector protein 3hb12 (to be used with hinge cs074 (SEQ |
| | ID NO: 1, 4 or 29)) |
| 38 | MSGGLEEEARELAEEAREVRRRAEELRRRAEEARETGEASEEHAAALLAEAAVLEL |
| | KAVLLELEARRLLKESGGEVAREALELAREARREAREALEAAEEASEGSGSHHWGS |
| | THHHHHH |
| | effector protein 3hb21 (to be used with hinge cs221 (SEQ |
| | ID NO: 3, 10, 19, 20, 31, or 32)) |
| 39 | MSGSKEEAKKEFMELARKKAKEIEENPEKARELAEEALKELEKKYEELKKAGVPEK |
| | EALALYVIALARVLIAKLAAEEGSGSHHWGSTHHHHHH |
| | effector protein 3hb24 (to be used with hinge cs230 (SEQ |
| | ID NO: 12)) |
| 40 | MSGMEERYERAERLGREVAELLRRGAPEEEIRRAIEELLRLLDSLPLPPAGSSETA |
| | RLIALAALAAAAELALDLGSGSHHWGSTHHHHHH |
| | effector protein 3hb28 |
| 41 | MSGMEEKKRRIRELVEEARRLVEEGAPLEEIEERVREFAEELREIAKEAGKNSELK |
| | LMVAAALLILAALIIEKRGSGSHHWGSTHHHHHH |
| | effector protein 3hb30 |
| 42 | MSGVEEKIKEAEELARRMREALKKYEETGDEKYKEEIEKLSERLRELTREILEKGN |
| | VEEKQKAVLLTAIRALIEIYLRLLEKEKGSGSHHWGSTHHHHHH |
| | effector protein 3hb32 (to be used with hinge cs269 (SEQ |
| | ID NO: 14)) |
| 43 | MSGEEEMKRRIRELEREALRRLDEAARTGSVEELDRVYREERRRFEELREELRREG |
| | LEGSEVDLLWLEALAVVEAAYI GARVVLELAGSGSHHWGSTHHHHHH |
| | effector peptide cs074B (to be used with hinge cs074 |
| | (SEQ ID NO: 1, 4 or 29) or swapped target hinge csw20 |
| | (SEQ ID NO: 18)) |
| 49 | SEEQEAARLLELAVEDLKLVLDALEKRR |
| | effector peptide cs094B (to be used with hinge cs094 |
| | (SEQ ID NO: 5)) |
| 50 | SEVEELVEEVEELIKLLEEMTKR |
| | effector peptide cs129B (to be used with hinge cs129 |
| | (SEQ ID NO: 6)) |
| 51 | DESRERLKEEVKRLIEQLQKKNKKQQK |
| | effector peptide cs201B (to be used with hinge cs201 |
| | (SEQ ID NO:2, 7 or 30)) |
| 52 | SILELAEEVAEEIKEAIRIAAKGIREGLKE |
| | effector peptide cs207B (to be used with hinge cs207 |
| | (SEQ ID NO: 8)) |
| 53 | MEEARKELTREMIEVLREIEEGFKERFE |
| | effector peptide cs217B (to be used with hinge cs217 |
| | (SEQ ID NO: 9)) |
| 54 | EERKKKLAREVVREARRLIERLAEEE |
| | effector peptide cs221B (to be used with hinge cs221 |
| | (SEQ ID NO: 3, 10, 19, 20, 31, or 32) or swapped target |
| | hinge csw13 (SEQ ID NO: 17) |
| 55 | EERKKELAKEVIETAKKLIEKLAKEE |
| | effector peptide cs230B (to be used with hinge cs230 |
| | (SEQ ID NO: 12)) |
| 56 | SSETAREIARVALEAAKWLYEDLKAEE |
| | effector peptide cs244B (to be used with hinge cs244) |
| 57 | SEEKLAEAARLLIEAAEIIKERGEKEG |
| | effector peptide cs245B (to be used with hinge cs245) |
| 58 | VEKLQREVLEEAERALREILERLEEKEK |
| | effector peptide cs253B (to be used with hinge cs253 |
| | (SEQ ID NO: 13)) |
| 59 | SATDKKLEDIALEMGGYLAVLMVDILKK |
| | effector peptide cs269B (to be used with hinge cs269 |
| | (SEQ ID NO: 14)) |
| 60 | SEELLRELEELAVDEARRIGAEVVRRLA |
| | effector peptide cs287B (to be used with hinge cs287 |
| | (SEQ ID NO: 15)) |
| 61 | EKEEITEELREVLREAERLAEEVLRILE |
| | effector peptide cs292B (to be used with hinge cs292 |
| | (SEQ ID NO: 16)) |
| 62 | EVIRLGFEVAERVEELLREAREIIEATA |
| | effector peptide js007B (to be used with hinge js007 |
| | (SEQ ID NO: 11)) |
| 63 | EEEEEVHREMQKAVRKALEAIKRILK |

In another aspect, the disclosure provides nucleic acids encoding the polypeptide of any embodiment of the polypeptides and fusion proteins of the first and second aspects of the disclosure. The nucleic acids may comprise RNA or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the present disclosure provides expression vectors comprising the nucleic acid of any aspect of the disclosure operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector.

In a further aspect, the present disclosure provides host cells that comprise the expression vectors, polypeptides, and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

The disclosure also provides kits, comprising:
(a) one or more polypeptide or fusion protein of any embodiment or combination of embodiments of the first aspect of the disclosure; and
(b) one or more polypeptide of any embodiment or combination of embodiments of the second aspect of the disclosure.

In various embodiments, the kits comprise on or more combinations selected from:
(a) a first polypeptide or fusion protein comprising the amino acid sequence of SEQ ID NO:1, 4, 18, or 29; and a second polypeptide comprising the amino acid sequence selected from the group consisting of SEQ ID NO:33-38, 49;
(b) a first polypeptide or fusion protein comprising the amino acid sequence of SEQ ID NO: 3, 10, 17, 19, 20, 31, or 32; and a second polypeptide comprising the amino acid sequence selected from the group consisting of SEQ ID NO:39 and 55;
(c) a first polypeptide or fusion protein comprising the amino acid sequence of SEQ ID NO:12; and a second polypeptide comprising the amino acid sequence selected from the group consisting of SEQ ID NO:40 and 56;
(d) a first polypeptide or fusion protein comprising the amino acid sequence of SEQ ID NO:14; and a second polypeptide comprising the amino acid sequence selected from the group consisting of SEQ ID NO:43 and 60;
(e) a first polypeptide or fusion protein comprising the amino acid sequence of SEQ ID NO:5; and a second polypeptide comprising the amino acid sequence of SEQ ID NO:50;
(f) a first polypeptide or fusion protein comprising the amino acid sequence of SEQ ID NO:6; and a second polypeptide comprising the amino acid sequence of SEQ ID NO:51;
(g) a first polypeptide or fusion protein comprising the amino acid sequence selected from SEQ ID NO:2, 7, and 30; and a second polypeptide comprising the amino acid sequence of SEQ ID NO:52;
(h) a first polypeptide or fusion protein comprising the amino acid sequence of SEQ ID NO:8; and a second polypeptide comprising the amino acid sequence of SEQ ID NO:53;
(i) a first polypeptide or fusion protein comprising the amino acid sequence of SEQ ID NO:9; and a second polypeptide comprising the amino acid sequence of SEQ ID NO:54;
(j) a first polypeptide or fusion protein comprising the amino acid sequence of SEQ ID NO:13; and a second polypeptide comprising the amino acid sequence of SEQ ID NO:59;
(k) a first polypeptide or fusion protein comprising the amino acid sequence of SEQ ID NO:14; and a second polypeptide comprising the amino acid sequence of SEQ ID NO:60;
(l) a first polypeptide or fusion protein comprising the amino acid sequence of SEQ ID NO:15; and a second polypeptide comprising the amino acid sequence of SEQ ID NO:61;
(m) a first polypeptide or fusion protein comprising the amino acid sequence of SEQ ID NO:16; and a second polypeptide comprising the amino acid sequence of SEQ ID NO:62; and
(n) a first polypeptide or fusion protein comprising the amino acid sequence of SEQ ID NO:11; and a second polypeptide comprising the amino acid sequence selected from the group consisting of SEQ ID NO:63;
wherein in the first polypeptide or fusion protein, (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted.

These combinations provide the specific polypeptide or fusion protein of the first aspect of the disclosure and the effector polypeptide of the second aspect that they bind to.

The disclosure also provides methods for using the polypeptides, fusion proteins, nucleic acids, and expression vectors of the disclosure, comprising any combination of steps as recited here. As disclosed in the examples, the hinge polypeptides of the disclosure populate two well-defined and structured conformational states, rather than adopting a heterogenous mixture of structures, and are broadly applicable to design of functional proteins. Like transistors in electronic circuits, the hinge proteins can be coupled to external outputs and inputs to create sensing devices. By way of non-limiting example, hinge polypeptides containing a disulfide that locks them in state X (for example, SEQ ID NO:19-20) couple the input “red/ox state” to the output “target binding,” where the target can be a peptide or a protein, and FRET-labeled hinges (for example, SEQ ID NO:29-31) couple the input “target binding” to the output “FRET signal.”

The polypeptides and fusion proteins of the disclosure can also be use to generate stimulus-responsive protein assemblies that switch between two well-defined shapes or oligomeric states in the presence of an effector, by incorporating the hinges as modular building blocks. Installing enzymatic sites in hinges (i.e.: fusion proteins comprising an enzymatic site as a functional domain) such that substrate binding favors one state and product release favors the other state enable fuel-driven conformational cycling, a crucial step towards the de novo design of molecular motors.

### Examples

In nature, proteins that switch between two conformations in response to environmental stimuli structurally transduce biochemical information in a manner analogous to how transistors control information flow in computing devices. Designing proteins with two distinct but fully structured conformations is a challenge for protein design as it requires sculpting an energy landscape with two distinct minima. Here we describe the design of “hinge” proteins that populate one designed state in the absence of ligand and a second designed state in the presence of ligand. X-ray crystallography, electron microscopy, double electron-electron resonance spectroscopy and binding measurements demonstrate that, despite the significant structural differences, the two states are designed with atomic level accuracy and that the conformational and binding equilibria are closely coupled.

While many naturally occurring proteins adopt single folded states, conformational changes between distinct protein states are crucial to the functions of enzymes, cell receptors, and molecular motors. The extent of these changes ranges from small rearrangements of secondary structure elements, to domain rearrangements, to fold-switching or metamorphic proteins that adopt completely different structures. In many cases, these conformational changes are triggered by “input” stimuli such as binding of a target molecule, post-translational modification, or change in pH. These changes in conformation can in turn result in “output” actions such as enzyme activation, target binding, or oligomerization; protein conformational changes can thus couple a specific input to a specific output. The generation of proteins that can switch between two quite different structural states is a difficult challenge for computational protein design, which usually aims to optimize a single, very stable conformation to be the global minimum of the folding energy landscape. Design of such proteins requires reframing the design paradigm towards optimizing for more than one minimum on the energy landscape, while simultaneously avoiding undesired off-target minima.

### Hinge Design Method

We set out to design proteins that can switch between two well-defined and fully structured conformations. To facilitate experimental characterization of the conformational change and to ensure compatibility with downstream applications, we imposed several additional requirements. First, the conformational change between the two states should be large, with some inter-residue distances changing by tens of angstroms between the two states. Second, the conformational change should not require global unfolding, which can be very slow. Third, neither of the two states should have substantial exposed patches of hydrophobic residues, which can compromise solubility. Fourth, the conformational change should be readily coupled to a range of inputs and outputs. Given that proteins are stabilized by hydrophobic cores, collectively achieving all of these properties in one protein system is challenging: protein conformations that differ considerably typically will have different sets of buried hydrophobic residues and require substantial structural rearrangements for interconversion.

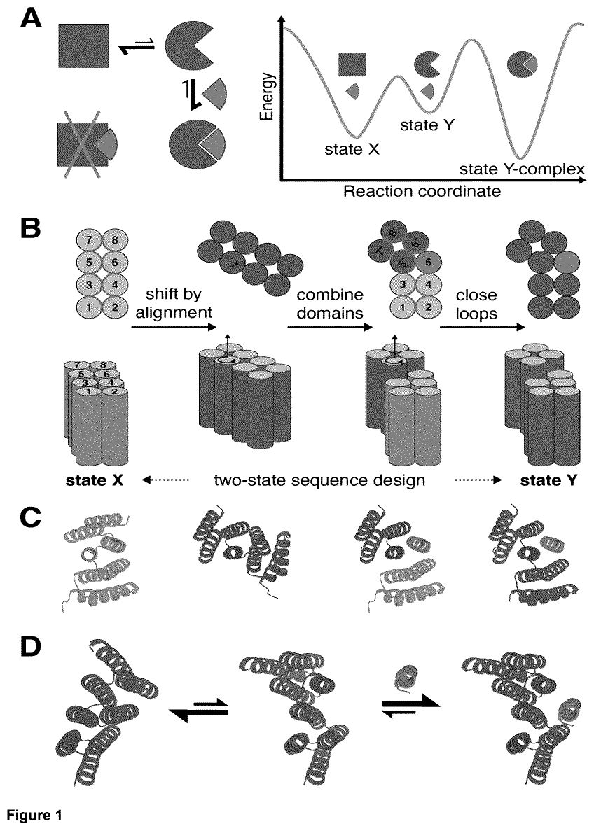

We reasoned that these goals could be collectively achieved with a “hinge”-like design in which two rigid domains move relative to each other while remaining individually folded. The hinge amplifies small local structural and chemical changes to achieve large global changes while the chemical environment for most residues remains similar throughout the conformational change, avoiding the need for global unfolding. Provided that the two states of the hinge bury similar sets of hydrophobic residues, the amount of exposed hydrophobic surface area can be kept low in both states. Designing one of the resulting conformations to bind to a target effector couples the conformational equilibrium with target binding (FIG. 1A). This design concept has precedent in nature; for example bacterial two-component systems utilize binding proteins that undergo hinging between two discrete conformations in response to ligand binding (20).

To implement this two-state hinge design concept, we took advantage of designed helical repeat proteins (DHRs, (21); FIG. 1B,C left) and DHR-based junction proteins (22). The backbone conformation of the DHR serves as the first conformational state of our hinge protein (“state X”). To generate a second conformation, a copy of the parent protein is rotated around a “pivot helix” (FIG. 1B,C) and a new backbone conformation is then created by combining the first half of the original protein (domain 1), the second half of the copy (domain 2), and either the helix following the pivot helix from the original protein or the helix preceding the pivot helix from the rotated copy (“peptide”). Rosetta™ FastDesign™ with backbone movement (23, 24) is used to re-design the interface between the three parts, and the two domains are connected into a single chain using fragment-based loop closure (21, 25, 26). Using a combination of Rosetta™ two-state design (see methods section for details) and ProteinMPNN™ (27) with linked residue identities, a single amino acid sequence is generated that is compatible with the state X hinge as well as with the state Y hinge-peptide complex. AlphaFold2™ (AF2)(28) with initial guess (29) is then used to predict the structure of the hinge with and without the effector peptide, allowing for the selection of designs that are predicted in the correct state X in absence of the peptide and in the correct state Y complex in presence of the peptide. To favor designs that are predominantly in the closed state in absence of the peptide (FIG. 1A,D), designs are selected only if state X has lower energy (computed using Rosetta™) than state Y in absence of the peptide, and if the state Y complex has lower energy than state X plus spatially separated peptide. Designs are also filtered on standard interface design metrics for the bound conformation (see Methods for details on filtering) (30).

Hinges Bind Effector Peptides with Sub-nM to Low μM Affinities

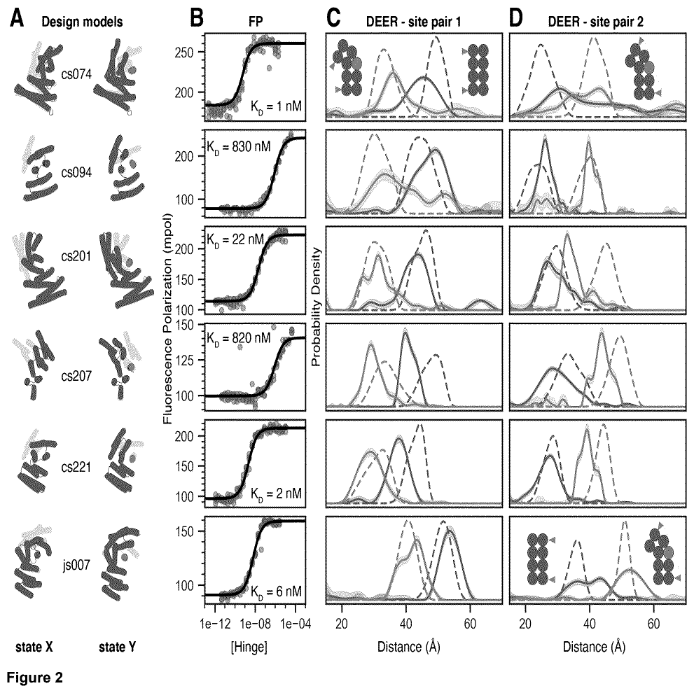

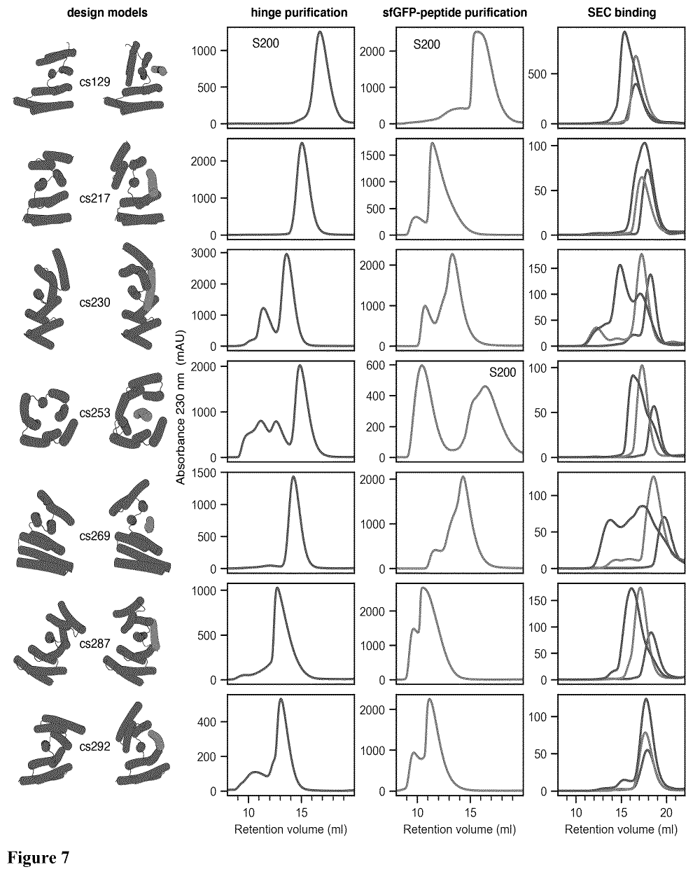

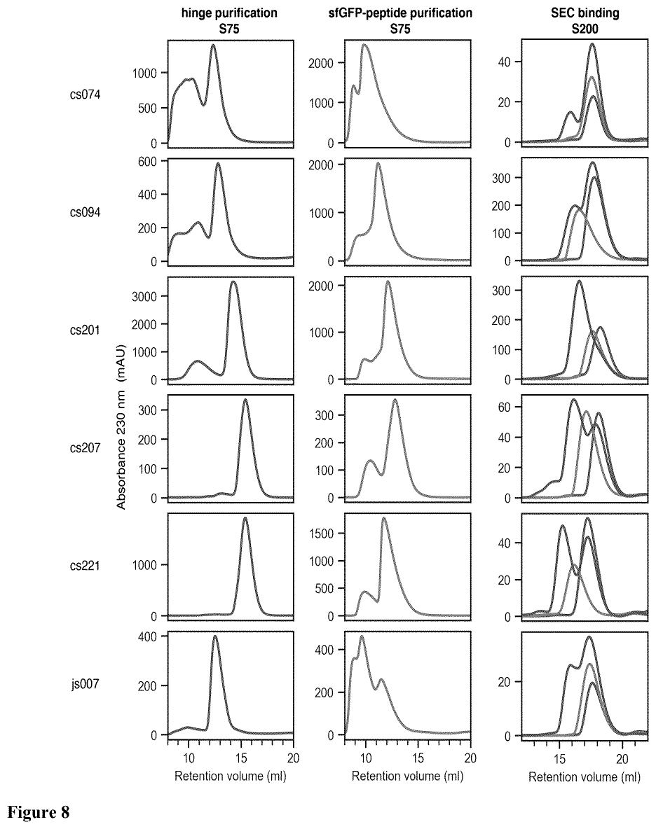

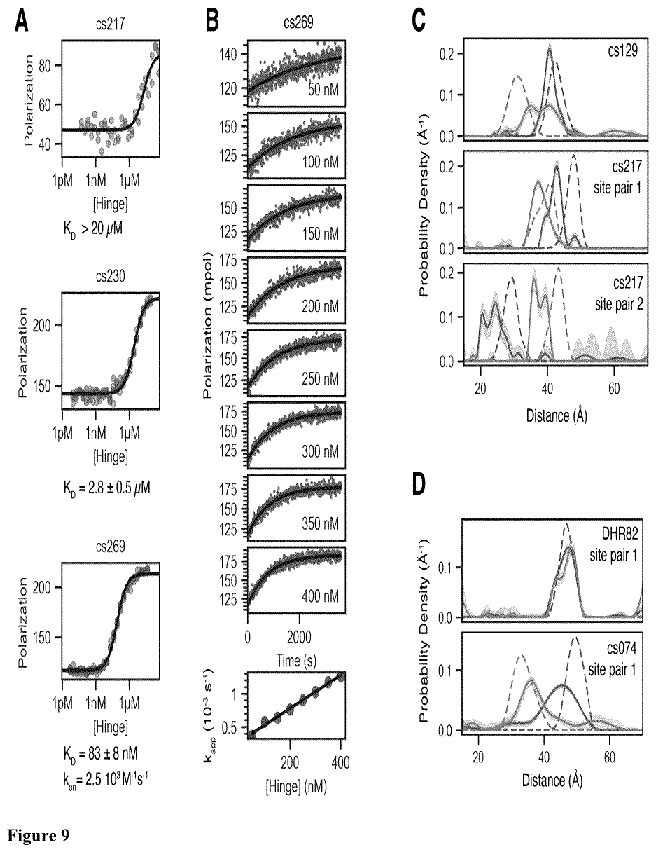

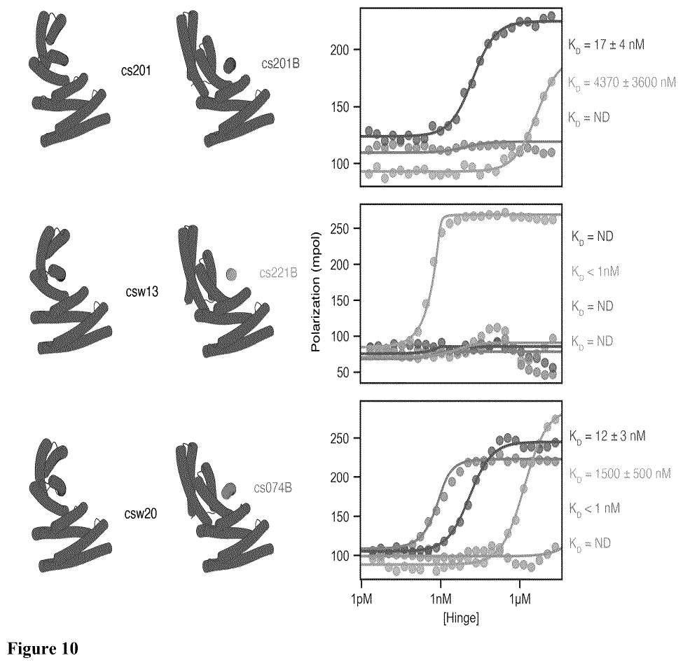

We used our hinge design approach to generate hinge-peptide pairs that span a wide structural space (FIGS. 1D, 2A, 6, 7). We experimentally tested multiple rounds of designs, using both DHRs (21) and helical junctions (22) as input scaffolds, and improving individual steps of the design pipeline between iterations. Designs for which hinge and GFP-fused peptide were soluble and interacted as judged by size exclusion chromatography (SEC, FIGS. 7-8) were selected for further characterization by fluorescence polarization (FP). Hinge-peptide binding affinities obtained from FP titration experiments with constant peptide concentration and varying hinge concentrations ranged from 1 nM to the low μM range (FIGS. 2B, 9, Table 5). To circumvent the bottleneck of finding soluble peptide sequences, we also sought to design hinges that bind to a given target peptide. Starting from design cs201, we used a modified version of our design pipeline to redesign the hinge to bind peptides cs074B or cs221B, respectively, which have similar hydrophobic fingerprints as the original target peptide cs201B. This one-sided two-state design approach yielded hinge designs that showed strong binding to their new target peptide while showing no or only weak off-target binding (FIG. 10).

### Effector Binding Modulates the Hinge Conformational Equilibrium

To characterize the conformational equilibrium of the designed hinges, we introduced two surface cysteine residues into the hinge protein and covalently labeled them with the nitroxide spin label MTSL (31). We then used double electron-electron resonance spectroscopy (DEER) to determine distance distributions between the two spin labels and compared these to simulated (32) distance distributions based on the state X and state Y design models. This experiment was performed on two different labeling site pairs for each design: one pair where the distance is predicted to decrease in the presence of peptide (FIGS. 2C, 9C, D) and the other where it is predicted to increase (FIGS. 2D, 9C, D). In the absence of the peptide, the observed distance distributions closely matched the state X simulations. In all cases the distances between the two pairs of probes shifted upon addition of peptide to better match the state Y simulations, suggesting that addition of effector peptide causes the conformational equilibrium to shift towards state Y as designed. For example, cs074 (site pair 1) showed a clear peak between 40 and 50 Å in absence of the peptide, and a peak between 30 and 40 Å in presence of the peptide, and both peaks agree well with the corresponding simulations (FIG. 2C, top row). In a control experiment using the static parent DHR protein of design cs074, the distance distributions with and without peptide were identical and matched both the simulation for the parent design model, which closely resembles state X, and the experimental distance distribution for state X of cs074 (FIG. 9D).

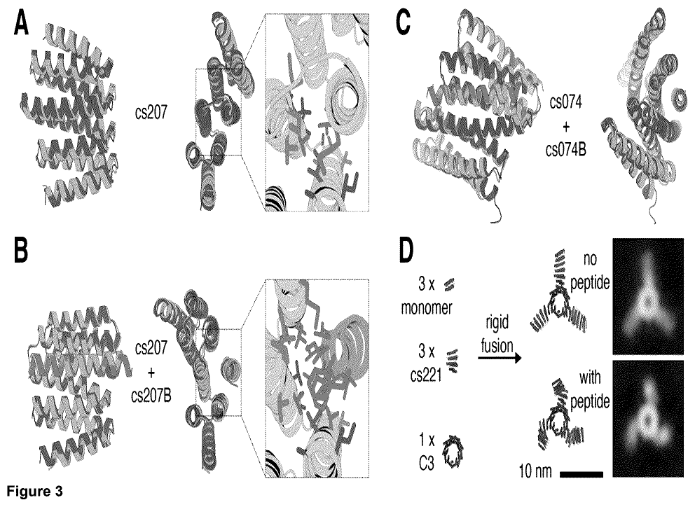

We solved crystal structures for two designs, cs207 and cs074. For design cs207, crystals were obtained from two separate crystallization screens: one screen for the hinge alone, and one screen for the hinge in complex with the target peptide. In the absence of peptide the experimental structure agrees well with the state X design model (FIG. 3A), and the structure of the hinge-peptide complex agrees well with the state Y design model (FIG. 3B). The crystal structures of hinge cs207 in both designed states demonstrate the accuracy with which two very different conformational states of the same protein can now be designed. For design cs074, the crystal structure of the hinge-peptide complex agrees well with the corresponding state Y design model (FIG. 3C).

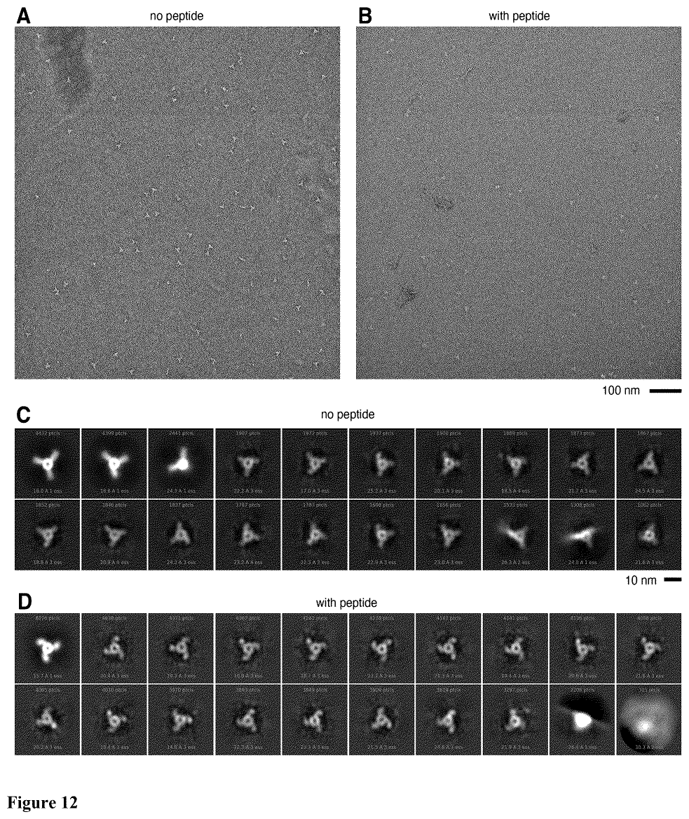

One major advantage of de novo designed proteins is their robustness to external conditions, such as high temperatures, and to structural perturbations, such as mutations, genetic fusion, and incorporation in designed protein assemblies. Circular Dichroism (CD) melts show that our hinges remain folded at high temperatures (FIG. 1), like the DHRs they were based on (21). To test whether our hinges can be incorporated as components of more complex protein assemblies without affecting their ability to undergo conformational changes, we designed a fully structured C3-symmetric protein with three hinge arms (FIG. 3D). We used inpainting (33) with RoseTTAFold™ (34) to rigidly connect one end of hinge cs221 to a previously validated homotrimer (35, 36) and the other end of the hinge to a previously validated monomeric protein (37). Negative-stain electron microscopy (nsEM) with reference-free class averaging shows straight arms in absence of peptide and bent arms in presence of peptide cs221B, corroborating the designed conformational change (FIGS. 3D, 12).

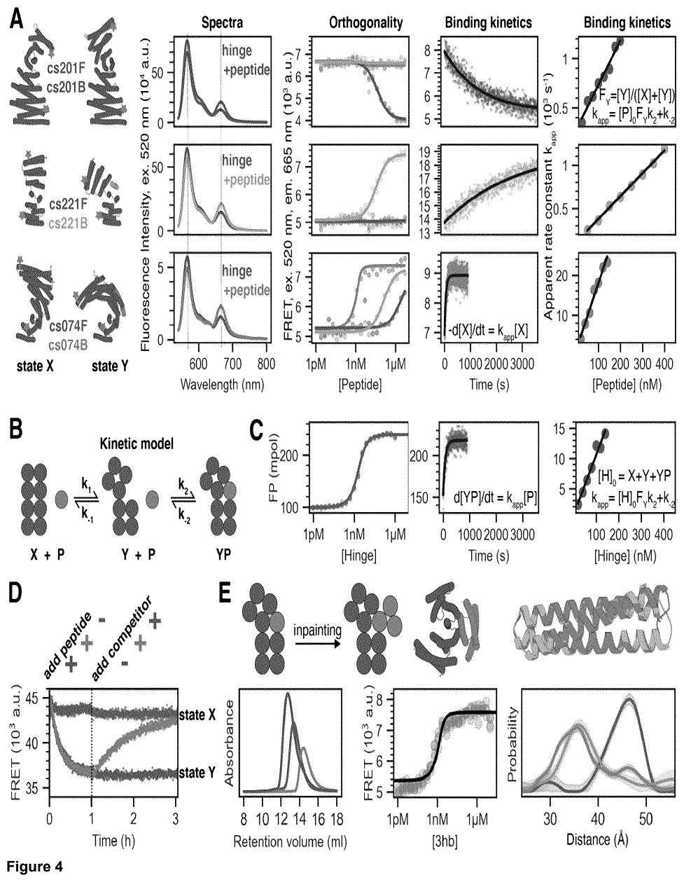

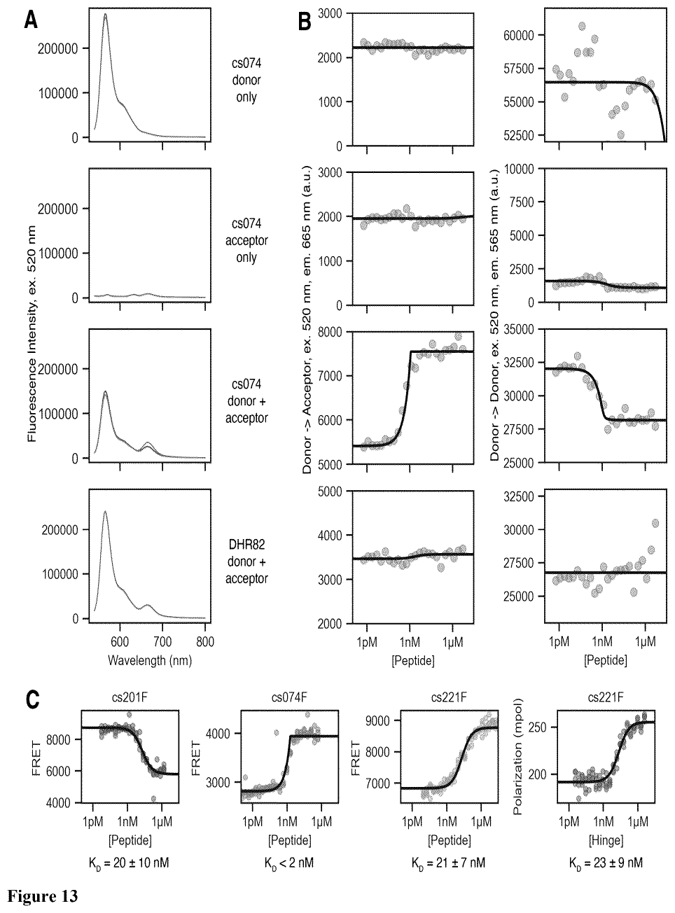

A critical feature of two-state switches in biology and technology is the coupling between the state control mechanism and the populations of the two states. To quantitatively investigate the thermodynamics and kinetics of the effector induced switching between the two states of our designed hinges, we used Forster resonance energy transfer (FRET). To increase both the absolute distance from N- to C-terminus and the change in termini distance between the two conformational states, we took advantage of the extensibility of repeat proteins and extended hinges cs074, cs221, and cs201 by 1-2 helices on their N and C termini, yielding cs074F, cs221F, and cs201F, respectively (FIG. 4A, first column). Single cysteines were introduced in helical regions near the termini of the extended hinges and stochastically labeled with an equal mixture of donor and acceptor dyes. For hinges cs074F and cs221F the distance between the label sites is above the R₀ of the dye pair in state X and below R₀ in state Y, and hence, acceptor emission upon donor excitation increases upon addition of the corresponding peptides cs074B and cs221B, respectively (FIG. 4A, second column). We used labeled, extended DHR82, the parent protein for cs074F, as a static control, and observed fluorescence spectra comparable to cs074F but no change in fluorescence upon addition of the peptide (FIG. 13A,B). For cs201F, the dye distance is above R₀ in state X and below R₀ in state Y, and donor emission decreases upon addition of peptide cs201B (FIG. 4A, second column). To test specificity of our hinge-peptide pairs, we performed pairwise titrations of all three labeled hinges at 2 nM with all three target peptides at varying concentrations. The on-target titrations had sigmoidal transitions that can be fitted with standard binding isotherms (FIGS. 4A, third column; 13C), whereas the off-target titrations for cs201F and cs221F show flat lines, indicating no conformational change of these hinges upon addition of off-target peptides at μM concentrations. cs074F showed weak off-target binding that was three orders of magnitude weaker for cs201B and two orders of magnitude weaker for cs221B compared to the on-target interaction for cs074B. cs201F and cs221F are thus orthogonal from the nM to the μM range, and the set of cs201F, cs221F, and cs074F is orthogonal over two orders of magnitude of effector concentration.

Association kinetics for the on-target interactions measured using constant concentrations of labeled hinge and varying excess concentrations of peptide are well fit by single exponentials (FIGS. 4A, fourth column; 14). The apparent rate constants increase linearly with increasing peptide concentration, exhibiting standard pseudo-first order kinetics for bimolecular reactions (FIGS. 4A, fifth column; 14). We analyze these data using a model comprising the three states (X, Y, Y+peptide) and four rate constants (FIG. 4B). The kinetic measurements using the FRET system follow the decrease in state X over time (d[X]/dt) upon the addition of peptide.

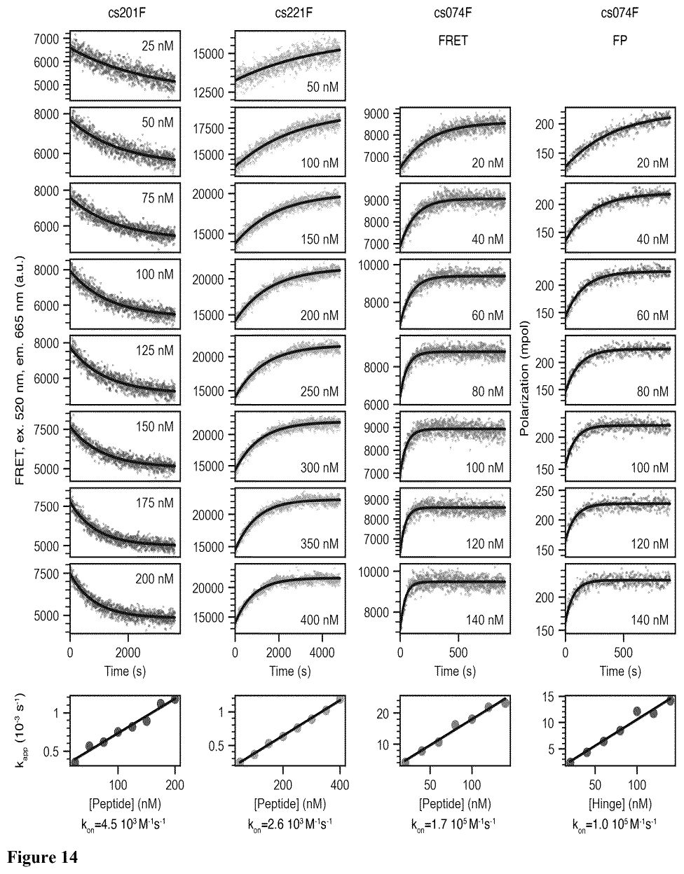

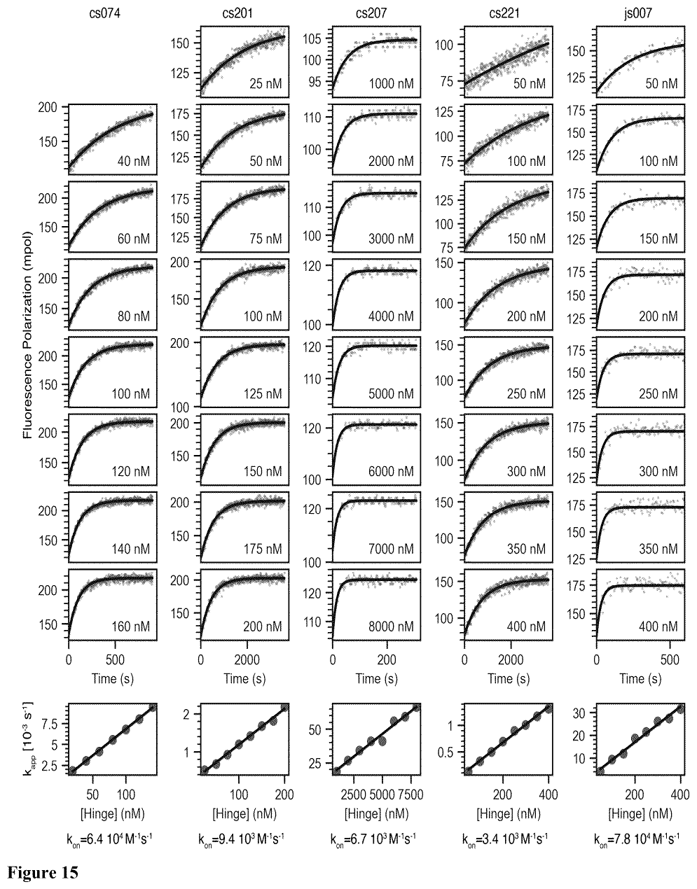

The observed pseudo-first order behavior (FIG. 4A, fifth column) indicates that the conformational change happens on a timescale that is faster than that of the observed binding and can be treated as a fast pre-equilibrium. The slopes of the linear pseudo-first order fits (kₒₙ) can thus be interpreted as the product of the microscopic association rate k₂ and the fractional population of state Y in absence of the peptide (F_{Y}=[Y]/([X]+[Y]). FP based titrations and kinetic characterization using the unlabeled extended hinge cs074F in excess over the TAMRA-labeled peptide cs074B agree well with the corresponding FRET experiments, further supporting the pre-equilibrium model (FIGS. 4C, 14). FP kinetics experiments for other hinge designs also follow pseudo-first order behavior with kₒₙ values ranging from 2.5×10³ M⁻¹s⁻¹ to 7.8×10⁴ M⁻¹s⁻¹ (FIGS. 9B, 15). To study the reversibility of hinge conformational changes, we started with 30 nM of FRET-labeled hinge cs201F (FIG. 4D), added 200 nM peptide to drive the conformational change, and then added excess unlabeled hinge cs201 to compete away the peptide. The FRET signal decreased upon addition of the peptide, consistent with conformational change from state X to state Y, and then returned to nearly the original level upon addition of unlabeled hinge, indicating that the hinge conformational change is fully reversible.

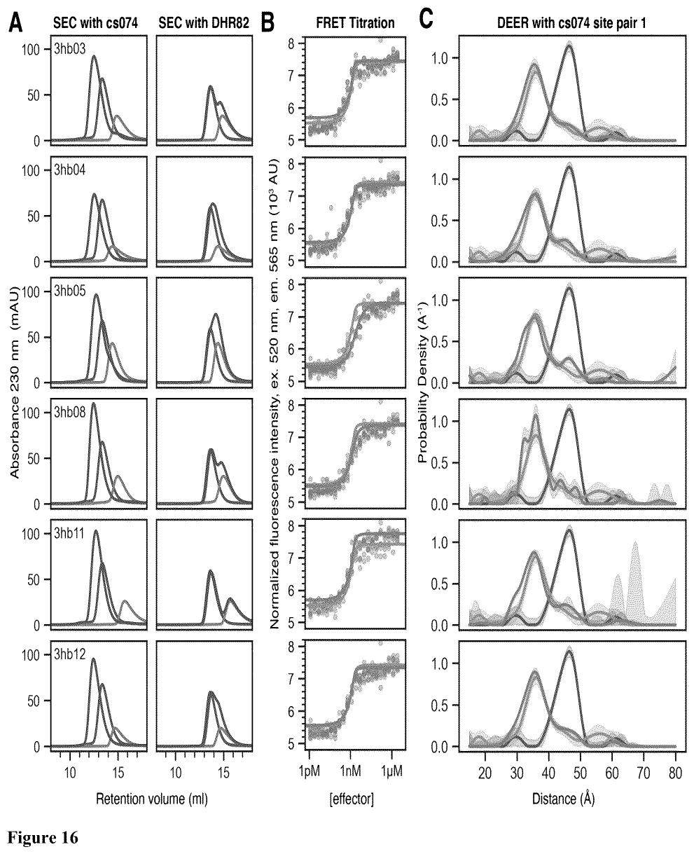

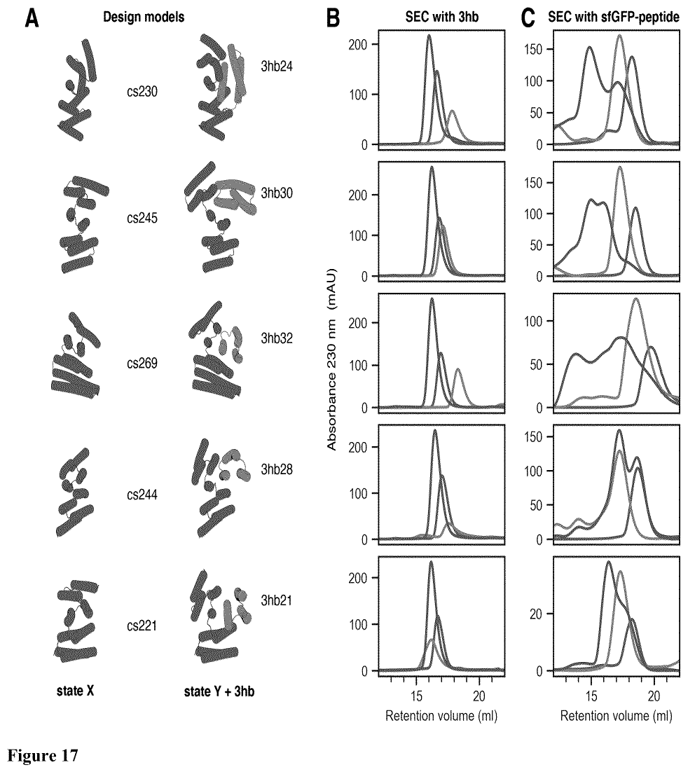

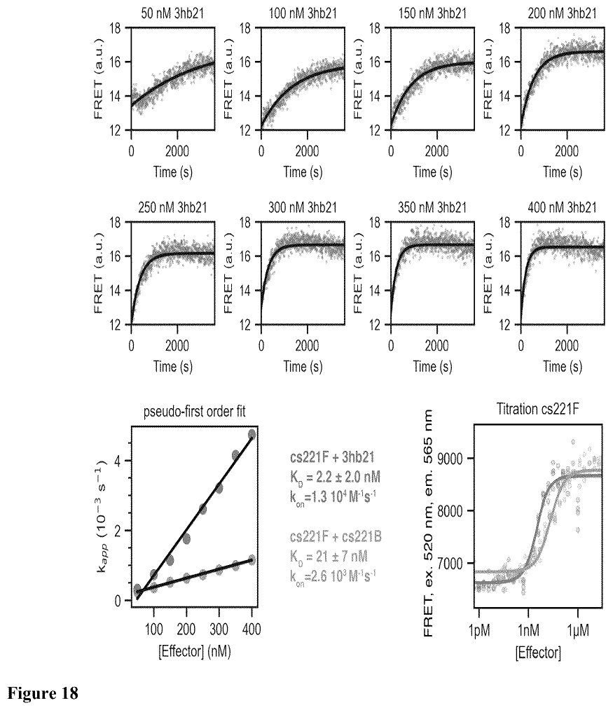

To explore whether peptide-responsive hinges could be turned into protein-responsive hinges, we used inpainting with RoseTTAFold™ to add two additional helices to a validated effector peptide, resulting in fully structured 3-helix bundles (3hb). For nine of our validated hinges we designed and experimentally characterized these effector proteins using SEC (FIGS. 4E, 16A, 17). Hinge-3hb binding was tested qualitatively by SEC and, for hinges which had a corresponding FRET construct, quantitatively with the FRET-labeled variant, and DEER was used in addition to FRET to confirm that 3hb binding caused the same conformational change as effector peptide binding (FIGS. 4E, bottom; 16). The affinity of 3hb05 to cs074F was similar to the affinity observed for the original peptide cs074B (FIG. 4E), whereas 3hb21 bound its target hinge cs221F significantly tighter than the original peptide cs221B (FIG. 18). The 3hb approach was able to rescue designs for which the peptide alone or the hinge-peptide complex had shown the tendency to form higher-order oligomers (FIG. 17). For two designs, 3hb05 and 3hb12, we obtained crystal structures that agreed well with the design models, indicating that the three-helix bundles are fully structured in isolation (FIG. 4E top right, 19).

### The Conformational Pre-Equilibrium Controls Effector Binding

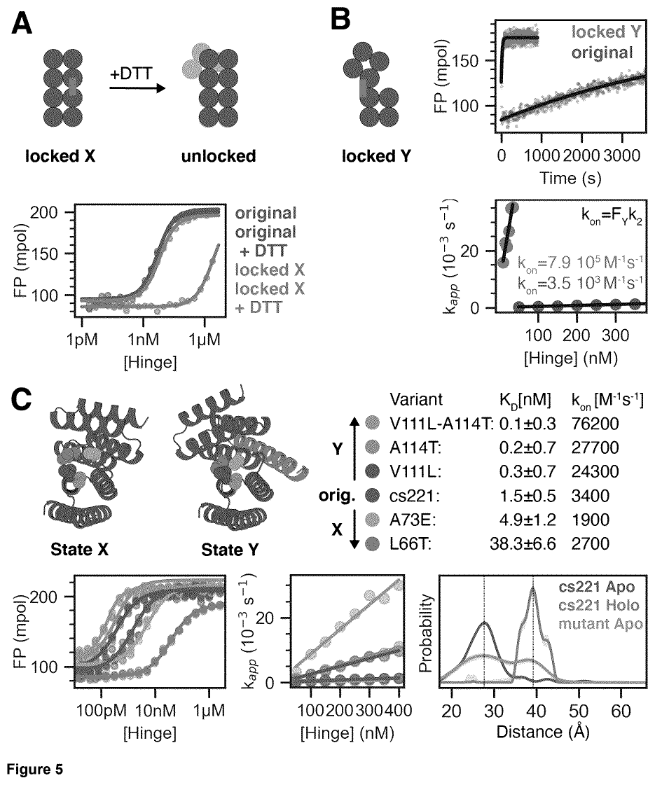

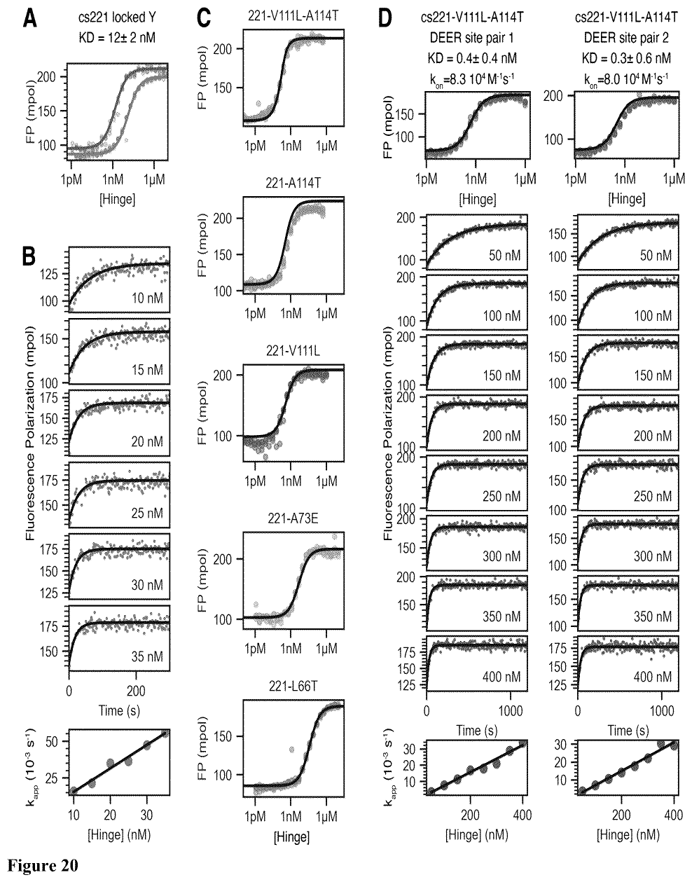

To test the effect of the conformational pre-equilibrium on effector binding, we introduced disulfide “staples” that lock the hinge in one conformation. Using FP we analyzed peptide binding to stapled versions of hinge cs221 (FIG. 5A,B). The variant that forms a disulfide bond in state X (“locked X”) showed only weak residual binding, likely due to a small fraction of hinges not forming the disulfide (FIG. 5A). Upon addition of the reducing agent dithiothreitol (DTT) to break the disulfide, peptide binding was fully restored, making this hinge variant a red/ox dependent peptide binder that binds the effector peptide under reducing but not under oxidizing conditions. The association rate for the locked Y variant was 200-fold higher than for the original hinge without disulfides (FIGS. 5B, 20A, B; despite this increase the overall binding affinity was weaker, suggesting the disulfide may lock the hinge in a slightly perturbed version of state Y). Using the pre-equilibrium model described above, the observed association rates provide an estimate of the fraction of hinge that is in state Y in absence of the peptide: a 200-fold higher observed on rate for the locked Y variant indicates a 200-fold higher fraction of hinge in state Y compared to the original hinge. Assuming that the locked Y variant is 100% in state Y and assuming that the microscopic rate constant k₂ is identical for the locked Y hinge and state Y of the original hinge, this would indicate that the original hinge is 99.5% in state X and 0.5% in state Y at equilibrium.

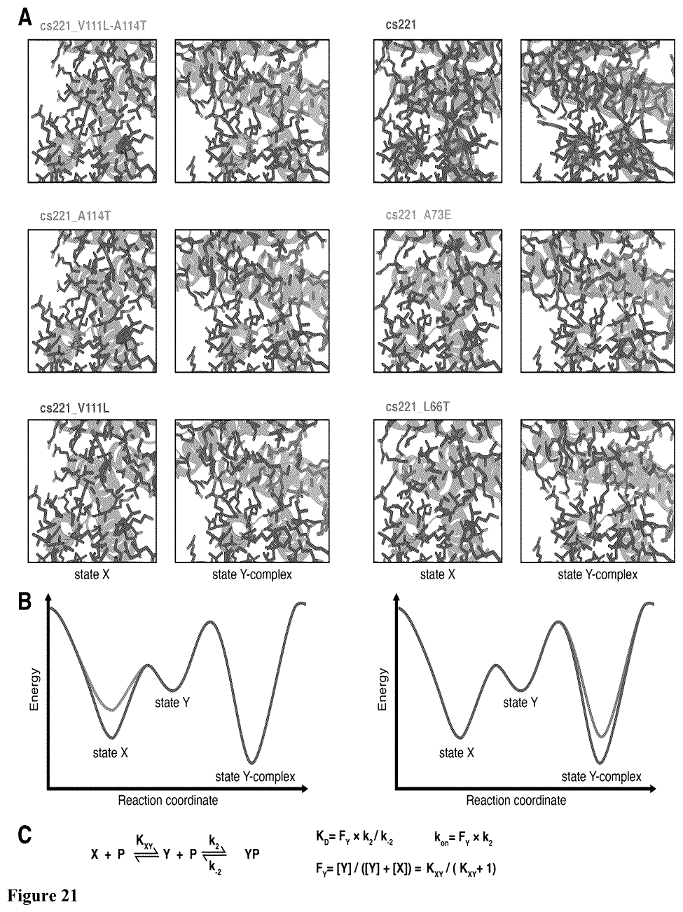

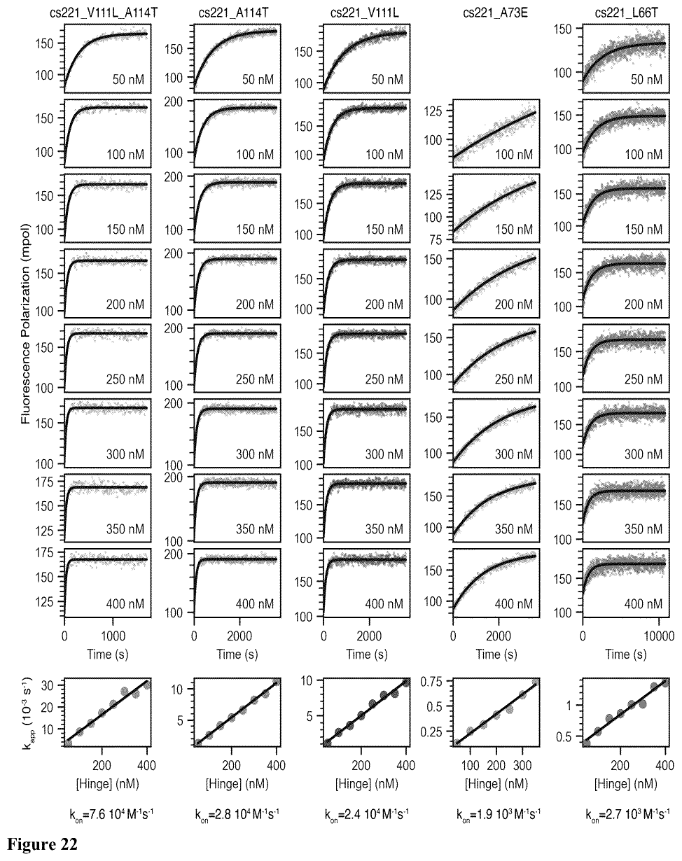

Having established the edge cases of locked state X and locked state Y, we sought to tune the pre-equilibrium by introducing single point mutations expected to specifically stabilize one state over the other while not directly affecting the peptide-binding interface. We used ProteinMPNN™ to generate consensus sequences (38) for each state and identified non-interface positions with distinct residue preferences that were different between both states (FIGS. 5C, 21A). We experimentally tested individual protein variants carrying substitutions expected to stabilize one state over the other without disrupting either conformation, as evaluated by AF2 predictions. Consistent with coupling of the conformational and binding equilibria, substitutions based on state X consensus sequences led to weaker peptide binding, and those based on state Y consensus sequences led to stronger binding (FIGS. 5C, 20C). The substitutions that stabilized state Y showed accelerated association kinetics (FIGS. 5C, 22), consistent with our kinetic model (FIGS. 4B, 21B, C): the mutations effectively shift the conformational pre-equilibrium towards state Y, increasing the on rates. This close coupling of the conformational equilibrium with association kinetics further supports the model outlined in FIG. 4B, and the fine tunability should be useful in downstream applications.

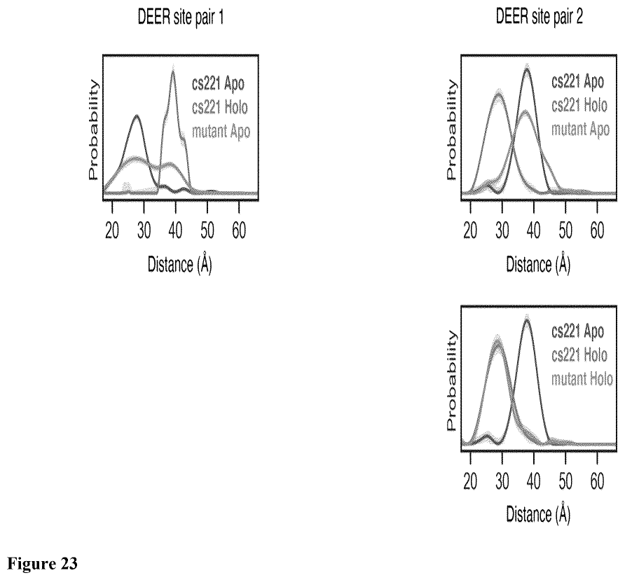

The state Y-stabilizing double mutant cs221_V111L_A114T has a 22-fold higher on rate than the original cs221, suggesting the occupancy of state Y in cs221_V111L_A114T is 22× higher in the absence of peptide. Distance distributions obtained from DEER measurements on site pair 2 of the double mutant cs221_V111L_A114T in absence of the peptide indeed showed an additional peak at a distance closely matching state Y (FIGS. 5C, 23). DEER measurements on site pair 1 of the double mutant showed a broader distribution with occupancy in the region corresponding to state Y (FIGS. 5C, 23). Measurements in the presence of the peptide were virtually indistinguishable from the original cs221 (FIG. 23). The double mutant thus populates two distinct states in the absence of the effector, and collapses to one state upon effector addition (FIGS. 5E, 23). The observation of a significant state Y population at equilibrium in the absence of the peptide as predicted based on the kinetic measurements further corroborates that the mutations affect the conformational pre-equilibrium, and provides strong support for our quantitative two-state model of the kinetics and thermodynamics of the designed hinge-effector systems.

### CONCLUSION

Our hinge design method generates proteins that populate two well-defined and structured conformational states, rather than adopting a heterogenous mixture of structures, and is broadly applicable to design of functional proteins. Like transistors in electronic circuits, we can couple the switches to external outputs and inputs to create sensing devices and incorporate them into larger protein systems to address a wide range of outstanding design challenges. Hinges containing a disulfide that locks them in state X couple the input “red/ox state” to the output “target binding,” where the target can be a peptide or a protein, and our FRET-labeled hinges couple the input “target binding” to the output “FRET signal.” Our approach can be readily extended such that state switching is driven by naturally occurring rather than designed effector peptides.

Stimulus-responsive protein assemblies that switch between two well-defined shapes or oligomeric states in the presence of an effector can now be built by incorporating the hinges as modular building blocks. Installing enzymatic sites in hinges such that substrate binding favors one state and product release favors the other state enable fuel-driven conformational cycling, a crucial step towards the de novo design of molecular motors. More generally, the ability to design two-state systems, and the designed two-state switches presented here, should enable protein design to go beyond static structures to more complex multistate assemblies and machines.

### REFERENCES

1. J. B. Stiller, R. Otten, D. Haussinger, P. S. Rieder, D. L. Theobald, D. Kern, Structure determination of high-energy states in a dynamic protein ensemble. Nature. 603, 528-535 (2022).
2. S. J. Kerns, R. V. Agafonov, Y.-J. Cho, F. Pontiggia, R. Otten, D. V. Pachov, S. Kutter, L. A. Phung, P. N. Murphy, V. Thai, T. Alber, M. F. Hagan, D. Kern, The energy landscape of adenylate kinase during catalysis. Nat. Struct. Mol. Biol. 22, 124-131 (2015).
3. A. Bisello, M. Chorev, M. Rosenblatt, L. Monticelli, D. F. Mierke, S. L. Ferrari, Selective ligand-induced stabilization of active and desensitized parathyroid hormone type 1 receptor conformations. J. Biol. Chem. 277, 38524-38530 (2002).
4. T. Movassagh, K. H. Bui, H. Sakakibara, K. Oiwa, T. Ishikawa, Nucleotide-induced global conformational changes of flagellar dynein arms revealed by in situ analysis. Nat. Struct. Mol. Biol. 17, 761-767 (2010).
5. W. A. Catterall, G. Wisedchaisri, N. Zheng, The conformational cycle of a prototypical voltage-gated sodium channel. Nat. Chem. Biol. 16, 1314-1320 (2020).
6. E. G. B. Evans, J. L. W. Morgan, F. DiMaio, W. N. Zagotta, S. Stoll, Allosteric conformational change of a cyclic nucleotide-gated ion channel revealed by DEER spectroscopy. Proc. Natl. Acad. Sci. U.S.A 117, 10839-10847 (2020).
7. B. Arragain, G. Effantin, P. Gerlach, J. Reguera, G. Schoehn, S. Cusack, H. Malet, Pre-initiation and elongation structures of full-length La Crosse virus polymerase reveal functionally important conformational changes. Nat. Commun. 11, 3590 (2020).
8. A. K. Kim, L. L. Porter, Functional and Regulatory Roles of Fold-Switching Proteins. Structure. 29, 6-14 (2021).
9. J.-H. Ha, S. N. Loh, Protein conformational switches: from nature to design. Chemistry. 18, 7984-7999 (2012).
10. P.-S. Huang, G. Oberdorfer, C. Xu, X. Y. Pei, B. L. Nannenga, J. M. Rogers, F. DiMaio, T. Gonen, B. Luisi, D. Baker, High thermodynamic stability of parametrically designed helical bundles. Science. 346, 481-485 (2014).
11. R. Koga, M. Yamamoto, T. Kosugi, N. Kobayashi, T. Sugiki, T. Fujiwara, N. Koga, Robust folding of a de novo designed ideal protein even with most of the core mutated to valine. Proc. Natl. Acad. Sci. U.S.A 117, 31149-31156 (2020).
12. A. F. Dishman, B. F. Volkman, Design and discovery of metamorphic proteins. Curr. Opin. Struct. Biol. 74, 102380 (2022).
13. N. H. Joh, T. Wang, M. P. Bhate, R. Acharya, Y. Wu, M. Grabe, M. Hong, G. Grigoryan, W. F. De Grado, De novo design of a transmembrane Zn2+-transporting four-helix bundle.
14. J. A. Davey, A. M. Damry, N. K. Goto, R. A. Chica, Rational design of proteins that exchange on functional timescales. Nat. Chem. Biol. 13, 1280-1285 (2017).
15. V. K. Mulligan, C. S. Kang, M. R. Sawaya, S. Rettie, X. Li, I. Antselovich, T. W. Craven, A. M. Watkins, J. W. Labonte, F. DiMaio, T. O. Yeates, D. Baker, Computational design of mixed chirality peptide macrocycles with internal symmetry. Protein Sci. 29, 2433-2445 (2020).
16. K. Y. Wei, D. Moschidi, M. J. Bick, S. Nerli, A. C. McShan, L. P. Carter, P.-S. Huang, D. A. Fletcher, N. G. Sgourakis, S. E. Boyken, D. Baker, Computational design of closely related proteins that adopt two well-defined but structurally divergent folds. Proc. Natl. Acad. Sci. U.S.A 117, 7208-7215 (2020).
17. R. A. Langan, S. E. Boyken, A. H. Ng, J. A. Samson, G. Dods, A. M. Westbrook, T. H. Nguyen, M. J. Lajoie, Z. Chen, S. Berger, V. K. Mulligan, J. E. Dueber, W. R. P. Novak, H. El-Samad, D. Baker, De novo design of bioactive protein switches. Nature. 572, 205-210 (2019).
18. A. Quijano-Rubio, H.-W. Yeh, J. Park, H. Lee, R. A. Langan, S. E. Boyken, M. J. Lajoie, L. Cao, C. M. Chow, M. C. Miranda, J. Wi, H. J. Hong, L. Stewart, B.-H. Oh, D. Baker, De novo design of modular and tunable protein biosensors. Nature. 591, 482-487 (2021).
19. J. Z. Zhang, H.-W. Yeh, A. C. Walls, B. I. M. Wicky, K. R. Sprouse, L. A. VanBlargan, R. Treger, A. Quijano-Rubio, M. N. Pham, J. C. Kraft, I. C. Haydon, W. Yang, M. DeWitt, J. E. Bowen, C. M. Chow, L. Carter, R. Ravichandran, M. H. Wener, L. Stewart, D. Veesler, M. S. Diamond, A. L. Greninger, D. M. Koelle, D. Baker, Thermodynamically coupled biosensors for detecting neutralizing antibodies against SARS-CoV-2 variants. Nat. Biotechnol. 40, 1336-1340 (2022).
20. M. Wang, Q. Guo, K. Zhu, B. Fang, Y. Yang, M. Teng, X. Li, Y. Tao, Interface switch mediates signal transmission in a two-component system. Proceedings of the National Academy of Sciences. 117, 30433-30440 (2020).
21. T. J. Brunette, F. Parmeggiani, P.-S. Huang, G. Bhabha, D. C. Ekiert, S. E. Tsutakawa, G. L. Hura, J. A. Tainer, D. Baker, Exploring the repeat protein universe through computational protein design. Nature. 528, 580-584 (2015).
22. T. J. Brunette, M. J. Bick, J. M. Hansen, C. M. Chow, J. M. Kollman, D. Baker, Modular repeat protein sculpting using rigid helical junctions. Proc. Natl. Acad. Sci. U.S.A 117, 8870-8875 (2020).
23. F. Khatib, S. Cooper, M. D. Tyka, K. Xu, I. Makedon, Z. Popovic, D. Baker, F. Players, Algorithm discovery by protein folding game players. Proc. Natl. Acad. Sci. U.S.A 108, 18949-18953 (2011).
24. M. D. Tyka, D. A. Keedy, I. André, F. Dimaio, Y. Song, D. C. Richardson, J. S. Richardson, D. Baker, Alternate states of proteins revealed by detailed energy landscape mapping. J. Mol. Biol. 405, 607-618 (2011).
25. N. Koga, R. Tatsumi-Koga, G. Liu, R. Xiao, T. B. Acton, G. T. Montelione, D. Baker, Principles for designing ideal protein structures. Nature. 491, 222-227 (2012).
26. Y.-R. Lin, N. Koga, R. Tatsumi-Koga, G. Liu, A. F. Clouser, G. T. Montelione, D. Baker, Control over overall shape and size in de novo designed proteins. Proc. Natl. Acad. Sci. U.S.A. 112, E5478-85 (2015).
27. J. Dauparas, I. Anishchenko, N. Bennett, H. Bai, R. J. Ragotte, L. F. Milles, B. I. M. Wicky, A. Courbet, R. J. de Haas, N. Bethel, P. J. Y. Leung, T. F. Huddy, S. Pellock, D. Tischer, F. Chan, B. Koepnick, H. Nguyen, A. Kang, B. Sankaran, A. K. Bera, N. P. King, D. Baker, Robust deep learning-based protein sequence design using ProteinMPNN. Science. 378, 49-56 (2022).
28. J. Jumper, R. Evans, A. Pritzel, T. Green, M. Figurnov, O. Ronneberger, K. Tunyasuvunakool, R. Bates, A. Zidek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
29. N. Bennett, B. Coventry, I. Goreshnik, B. Huang, A. Allen, D. Vafeados, Y. P. Peng, J. Dauparas, M. Baek, L. Stewart, F. DiMaio, S. De Munck, S. N. Savvides, D. Baker, Improving de novo Protein Binder Design with Deep Learning. bioRxiv (2022), p. 2022.06.15.495993, doi:10.1101/2022.06.15.495993.
30. L. Cao, B. Coventry, I. Goreshnik, B. Huang, W. Sheffler, J. S. Park, K. M. Jude, I. Marković, R. U. Kadam, K. H. G. Verschueren, K. Verstraete, S. T. R. Walsh, N. Bennett, A. Phal, A. Yang, L. Kozodoy, M. DeWitt, L. Picton, L. Miller, E.-M. Strauch, N. D. DeBouver, A. Pires, A. K. Bera, S. Halabiya, B. Hammerson, W. Yang, S. Bernard, L. Stewart, I. A. Wilson, H. Ruohola-Baker, J. Schlessinger, S. Lee, S. N. Savvides, K. C. Garcia, D. Baker, Design of protein-binding proteins from the target structure alone. Nature. 605, 551-560 (2022).
31. Berliner, Olga Hankovszky, A Novel Reversible Thiol-Specific Spin Label: Papain Active Site Labeling and Inhibition’. Anal. Biochem.
32. M. H. Tessmer, S. Stoll, chiLife: An open-source Python package for in silico spin labeling and integrative protein modeling. bioRxiv (2022), p. 2022.12.23.521725, doi:10.1101/2022.12.23.521725.
33. J. Wang, S. Lisanza, D. Juergens, D. Tischer, J. L. Watson, K. M. Castro, R. Ragotte, A. Saragovi, L. F. Milles, M. Baek, I. Anishchenko, W. Yang, D. R. Hicks, M. Expósit, T. Schlichthaerle, J.-H. Chun, J. Dauparas, N. Bennett, B. I. M. Wicky, A. Muenks, F. DiMaio, B. Correia, S. Ovchinnikov, D. Baker, Scaffolding protein functional sites using deep learning. Science. 377, 387-394 (2022).
34. M. Baek, F. DiMaio, I. Anishchenko, J. Dauparas, S. Ovchinnikov, G. R. Lee, J. Wang, Q. Cong, L. N. Kinch, R. D. Schaeffer, C. Millan, H. Park, C. Adams, C. R. Glassman, A. DeGiovanni, J. H. Pereira, A. V. Rodrigues, A. A. van Dijk, A. C. Ebrecht, D. J. Opperman, T. Sagmeister, C. Buhlheller, T. Pavkov-Keller, M. K. Rathinaswamy, U. Dalwadi, C. K. Yip, J. E. Burke, K. C. Garcia, N. V. Grishin, P. D. Adams, R. J. Read, D. Baker, Accurate prediction of protein structures and interactions using a three-track neural network. Science. 373, 871-876 (2021).
35. J. P. Hallinan, L. A. Doyle, B. W. Shen, M. M. Gewe, B. Takushi, M. A. Kennedy, D. Friend, J. M. Roberts, P. Bradley, B. L. Stoddard, Design of functionalised circular tandem repeat proteins with longer repeat topologies and enhanced subunit contact surfaces. Commun Biol. 4, 1240 (2021).
36. R. D. Kibler, S. Lee, M. A. Kennedy, B. I. M. Wicky, S. M. Lai, M. M. Kostelic, X. Li, C. M. Chow, L. Carter, V. H. Wysocki, B. L. Stoddard, D. Baker, Stepwise design of pseudosymmetric protein hetero-oligomers. bioRxiv (2023), p. 2023.04.07.535760, doi:10.1101/2023.04.07.535760.
37. D. D. Sahtoe, F. Praetorius, A. Courbet, Y. Hsia, B. I. M. Wicky, N. I. Edman, L. M. Miller, B. J. R. Timmermans, J. Decarreau, H. M. Morris, A. Kang, A. K. Bera, D. Baker, Reconfigurable asymmetric protein assemblies through implicit negative design. Science. 375, eabj7662 (2022).
38. G. E. Crooks, G. Hon, J.-M. Chandonia, S. E. Brenner, WebLogo: a sequence logo generator. Genome Res. 14, 1188-1190 (2004).
39. K. Wu, H. Bai, Y.-T. Chang, R. Redler, K. E. McNally, W. Sheffler, T. J. Brunette, D. R. Hicks, T. E. Morgan, T. J. Stevens, A. Broerman, I. Goreshnik, M. DeWitt, C. M. Chow, Y. Shen, L. Stewart, E. Derivery, D. A. Silva, G. Bhabha, D. C. Ekiert, D. Baker, De novo design of modular peptide-binding proteins by superhelical matching. Nature (2023), doi:10.1038/s41586-023-05909-9.
40. S. V. Torres, P. J. Y. Leung, I. D. Lutz, P. Venkatesh, J. L. Watson, F. Hink, H.-H. Huynh, A. H.-W. Yeh, D. Juergens, N. R. Bennett, A. N. Hoofnagle, E. Huang, M. J. MacCoss, M. Expósit, G. R. Lee, E. N. Korkmaz, J. Nivala, L. Stewart, J. M. Rodgers, D. Baker, De novo design of high-affinity protein binders to bioactive helical peptides. bioRxiv (2022), p. 2022.12.10.519862, doi:10.1101/2022.12.10.519862.
41. Florian Praetorius, Philip J. Y. Leung, Maxx H. Tessmer, Adam Broerman, Cullen Demakis, Abbas Idris, Stefan Stoll, and David Baker, Data for Design of stimulus-responsive two-state hinge proteins. Zenodo, doi:10.5281/zenodo.8122343.
42. R. F. Alford, A. Leaver-Fay, J. R. Jeliazkov, M. J. O'Meara, F. P. DiMaio, H. Park, M. V. Shapovalov, P. D. Renfrew, V. K. Mulligan, K. Kappel, J. W. Labonte, M. S. Pacella, R. Bonneau, P. Bradley, R. L. Dunbrack Jr, R. Das, D. Baker, B. Kuhlman, T. Kortemme, J. J. Gray, The Rosetta All-Atom Energy Function for Macromolecular Modeling and Design. J. Chem. Theory Comput. 13, 3031-3048 (2017).
43. L. An, G. R. Lee, De Novo Protein Design Using the Blueprint Builder in Rosetta. Curr. Protoc. Protein Sci. 102, e116 (2020).
44. M. F. Sauer, A. M. Sevy, J. E. Crowe Jr, J. Meiler, Multi-state design of flexible proteins predicts sequences optimal for conformational change. PLoS Comput. Biol. 16, e1007339 (2020).
45. N. Chennamsetty, V. Voynov, V. Kayser, B. Helk, B. L. Trout, Design of therapeutic proteins with enhanced stability. Proc. Natl. Acad. Sci. U.S.A 106, 11937-11942 (2009).
46. S. Warszawski, R. Netzer, D. S. Tawfik, S. J. Fleishman, A “fuzzy”-logic language for encoding multiple physical traits in biomolecules. J. Mol. Biol. 426, 4125-4138 (2014).
47. S. T. Kudlacek, S. Metz, D. Thiono, A. M. Payne, T. T. N. Phan, S. Tian, L. J. Forsberg, J. Maguire, I. Seim, S. Zhang, A. Tripathy, J. Harrison, N. I. Nicely, S. Soman, M. K. McCracken, G. D. Gromowski, R. G. Jarman, L. Premkumar, A. M. de Silva, B. Kuhlman, Designed, highly expressing, thermostable dengue virus 2 envelope protein dimers elicit quaternary epitope antibodies. Sci. Adv. 7, eabg4084 (2021).
48. B. I. M. Wicky, L. F. Milles, A. Courbet, R. J. Ragotte, J. Dauparas, E. Kinfu, S. Tipps, R. D. Kibler, M. Baek, F. DiMaio, X. Li, L. Carter, A. Kang, H. Nguyen, A. K. Bera, D. Baker, Hallucinating symmetric protein assemblies. Science. 378, 56-61 (2022).
49. M. Mirdita, K. Schiitze, Y. Moriwaki, L. Heo, S. Ovchinnikov, M. Steinegger, ColabFold: making protein folding accessible to all. Nat. Methods. 19, 679-682 (2022).
50. T. M. Lauer, N. J. Agrawal, N. Chennamsetty, K. Egodage, B. Helk, B. L. Trout, Developability index: a rapid in silico tool for the screening of antibody aggregation propensity. J. Pharm. Sci. 101, 102-115 (2012).
51. S. Yao, A. Moyer, Y. Zheng, Y. Shen, X. Meng, C. Yuan, Y. Zhao, H. Yao, D. Baker, C. Wu, De novo design and directed folding of disulfide-bridged peptide heterodimers. Nat. Commun. 13, 1539 (2022).
52. B. Dang, M. Mravic, H. Hu, N. Schmidt, B. Mensa, W. F. DeGrado, SNAC-tag for sequence-specific chemical protein cleavage. Nat. Methods. 16, 319-322 (2019).
53. P. Virtanen, R. Gommers, T. E. Oliphant, M. Haberland, T. Reddy, D. Cournapeau, E. Burovski, P. Peterson, W. Weckesser, J. Bright, S. J. van der Walt, M. Brett, J. Wilson, K. J. Millman, N. Mayorov, A. R. J. Nelson, E. Jones, R. Kern, E. Larson, C. J. Carey, I. Polat, Y. Feng, E. W. Moore, J. VanderPlas, D. Laxalde, J. Perktold, R. Cimrman, I. Henriksen, E. A. Quintero, C. R. Harris, A. M. Archibald, A. H. Ribeiro, F. Pedregosa, P. van Mulbregt, SciPy 1.0 Contributors, SciPy 1.0: fundamental algorithms for scientific computing in Python. Nat. Methods. 17, 261-272 (2020).
54. M. H. Tessmer, E. R. Canarie, S. Stoll, Comparative evaluation of spin-label modeling methods for protein structural studies. Biophys. J. 121, 3508-3519 (2022).
55. Y. Polyhach, E. Bordignon, G. Jeschke, Rotamer libraries of spin labelled cysteines for protein studies. Phys. Chem. Chem. Phys. 13, 2356-2366 (2011).
56. S. Stoll, A. Schweiger, EasySpin, a comprehensive software package for spectral simulation and analysis in EPR. J. Magn. Reson. 178, 42-55 (2006).
57. L. Fibregas Ibinez, G. Jeschke, S. Stoll, DeerLab: a comprehensive software package for analyzing dipolar electron paramagnetic resonance spectroscopy data. Magn Reson (Gott). 1, 209-224 (2020).
58. L. Fibregas-Ibinez, G. Jeschke, S. Stoll, Compactness regularization in the analysis of dipolar EPR spectroscopy data. J. Magn. Reson. 339, 107218 (2022).
59. W. Kabsch, XDS. Acta Crystallogr. D Biol. Crystallogr. 66, 125-132 (2010).
60. M. D. Winn, C. C. Ballard, K. D. Cowtan, E. J. Dodson, P. Emsley, P. R. Evans, R. M. Keegan, E. B. Krissinel, A. G. W. Leslie, A. McCoy, S. J. McNicholas, G. N. Murshudov, N. S. Pannu, E. A. Potterton, H. R. Powell, R. J. Read, A. Vagin, K. S. Wilson, Overview of the CCP4 suite and current developments. Acta Crystallogr. D Biol. Crystallogr. 67, 235-242 (2011).
61. A. J. McCoy, R. W. Grosse-Kunstleve, P. D. Adams, M. D. Winn, L. C. Storoni, R. J. Read, Phaser crystallographic software. J. Appl. Crystallogr. 40, 658-674 (2007).
62. P. D. Adams, P. V. Afonine, G. Bunkóczi, V. B. Chen, I. W. Davis, N. Echols, J. J. Headd, L.-W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger, P. H. Zwart, PHENIX: a comprehensive Python-based system for macromolecular structure solution. Acta Crystallogr. D Biol. Crystallogr. 66, 213-221 (2010).
63. P. Emsley, K. Cowtan, Coot: model-building tools for molecular graphics. Acta Crystallogr. D Biol. Crystallogr. 60, 2126-2132 (2004).
64. C. J. Williams, J. J. Headd, N. W. Moriarty, M. G. Prisant, L. L. Videau, L. N. Deis, V. Verma, D. A. Keedy, B. J. Hintze, V. B. Chen, S. Jain, S. M. Lewis, W. B. Arendall III, J. Snoeyink, P. D. Adams, S. C. Lovell, J. S. Richardson, D. C. Richardson, MolProbity: More and better reference data for improved all-atom structure validation. Protein Sci. 27, 293-315 (2018).

### Materials and Methods

### Generating and Pairing Hinge Conformational States

We used curated libraries of DHRs as inputs for generation of hinge conformations. The backbone conformation of a given DHR serves as a template for the first conformational state (“state X”) of the hinge. To generate a second conformation, we generated a copy of the parent protein and rotated it around a “pivot helix” by aligning the copy to the original DHR shifted by N residues, where −7<N<7 (FIG. 1B,C). We then created a new backbone conformation by combining the first half of the original protein (domain 1), the second half of the copy (domain 2), and either the helix following the pivot helix from the original protein or the helix preceding the pivot helix from the rotated copy (“peptide”). This alignment-based alternative state generation protocol was implemented using custom PyRosetta™ functions. To discard backbone arrangements with significant clashes, we mutate the entire backbone to glycine and score the resulting pose with only the Rosetta™ “fa_rep” scoreterm (42), set to a weight of 1.0, with the “beta_nov16” scorefunction. At this stage, the helical peptide was sometimes extended using a similar alignment/shifting strategy to increase the size of the interface in state Y. We used PyRosetta™ FastDesign™ with backbone and jump movement to further improve the backbone and sequence around the tripartite interface between the first hinge domain, the peptide, and the second hinge domain for scoring purposes. At this stage, we discarded any designs where either of the domains or the peptide didn't form good contacts with the other two chains using interface metrics (30). Using fragment-based loop closure (21, 25, 26), we connected domains 1 and 2 connected into a single chain that serves as the second conformational state of the hinge protein (“state Y”). We called the blueprint builder (43) in PyRosetta™ to rebuild the loop region between the hinge domains in state X based on a secondary structure template of the hinge in state Y. This procedure yields pairs of state X and state Y backbones with matching loop lengths and secondary structures.

### Two-State Sequence Design

Initially, we tried many different multi-state design (MSD) algorithms in Rosetta™. We first tried an approach where we would iterate between conformational states while performing single-state design (SSD) for each state individually while ramping a custom sequence convergence scoreterm between iterations (44). We found this method tended to decorate the surface with hydrophobics in positions that had ambiguous residue-level preferences between the conformational states, so we explicitly penalized excessive surface hydrophobics using constraints that calculated spatial aggregation propensity (SAP)(45) on the fly during design. We also used a quasisymmetric multistate design approach in PyRosetta™, performing design on both states simultaneously while forcing the packer to consider the chemical context of residue positions linked across the states (46). This method seemed to have fewer pathologies in terms of positional sequence selection but scaled poorly in terms of computational performance, so we chose not to use it for large-scale sequence design tasks. Ultimately, we extensively used FastDesign™ with a version of the annealer originally intended and optimized for multi-conformation, sequence-symmetric design (47), since it was the easiest to use and scaled well computationally while being easily tunable to avoid the pathologies of the iterative approach.

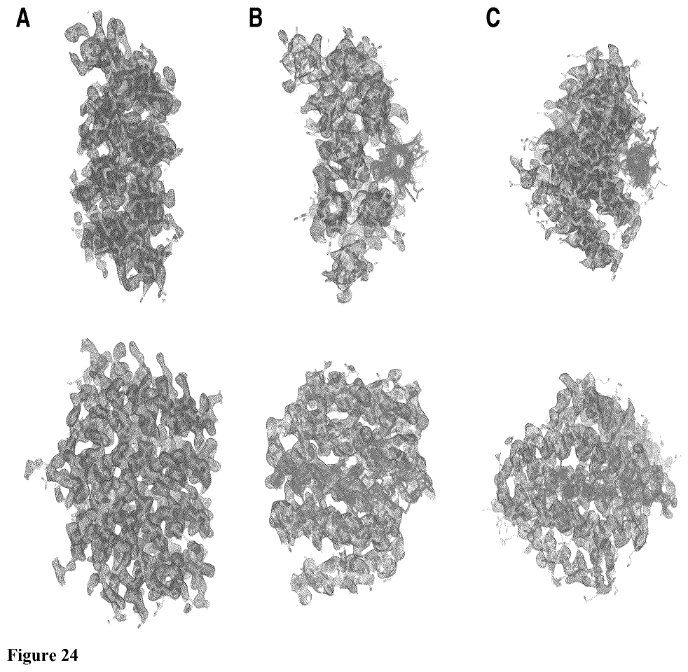

Once we had sampled sequences and backbones with Rosetta™ we optionally refined the sequences with ProteinMPNN™ (27) multistate design (MPNN-MSD). Using a feature intended for homooligomer symmetry (48) we tied corresponding residue positions probabilities together across chains and used MPNN to sample up to 96 sequences per pair of backbones. We then could use AF2 initial guess (AF2-IG)(29) to predict the structure of the effector-bound complex (state Y) by threading the MPNN-MSD sequences back onto the backbones using mean predicted Local Distance Difference Test scores (pLDDT), RMSD to reference design model, and mean off-diagonal Predicted Aligned Error matrix (PAE interaction) cutoffs of 93, 1.5, and 5, respectively for AF2. Designs that passed these criteria could be predicted again by AF2 (28, 49) to check if they folded to the correct closed position (state X) absent the effector sequence. We observed that sequences designed with MPNN-MSD had much better computational success rates and overall metrics (FIG. 24). After two-state design, most hinges were soluble and had significant monomer populations, however many of the effector peptides turned out to be insoluble or formed stable homooligomers. In some cases effector peptides were improved by simple redesign of the peptide surface residues away from the hinge interface, or by truncation of the peptide.

### Computational Filtering

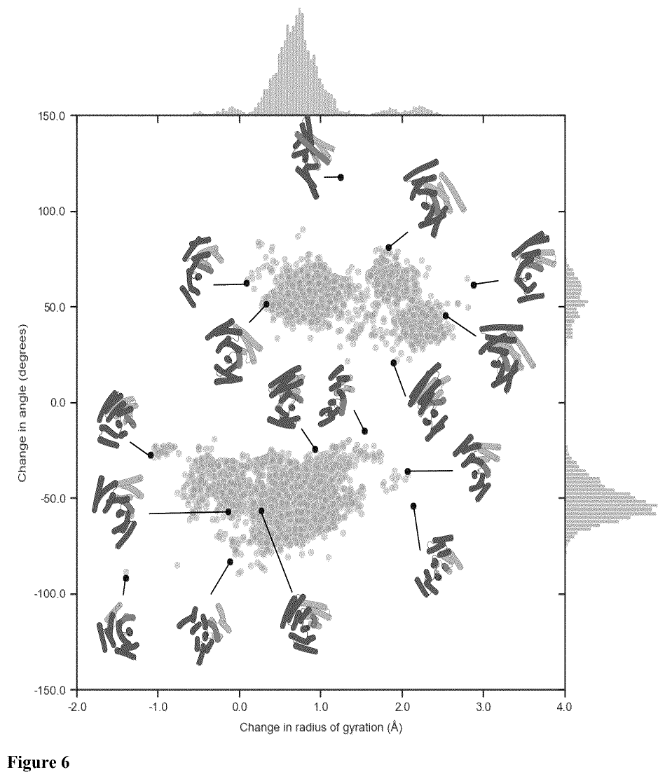

Concerned with the possibility that hinges designed with this process would randomly oscillate between closed and open conformations in the absence of the effector, we tried to implement additional filters to select only the designs that would have our intended behavior for testing. We chose designs where the hinge sequence scored more favorably in Rosetta™ in the closed conformation relative to the open conformation when the peptide was absent, but scored more favorably in the open conformation with the peptide bound in comparison to the sum of the scores of the closed conformation and the peptide alone. Similarly, we required that the solvent-exposed hydrophobicity, (measured by spatial aggregation propensity (SAP), would decrease in the closed conformation relative to the open conformation when the peptide was absent, and the bound complex would have less exposed hydrophobics compared to the sum of the exposed hydrophobics of the closed conformation and the peptide alone. We also filtered the bound conformation on interface design metrics, including ddG, cms and SASA. This pipeline for designing effector-binding hinges was able to generate very diverse outputs, with large differences in changes in shape and size (FIG. 6).

### One-Sided Two-State Design for Swapped Peptide Targets

To generate swapped-peptide designs, we started from the state X and state Y backbones of cs074, cs201, cs221, and js007, including peptide backbones. Peptide sequences were replaced, using the sequences of cs074B, cs201B, and cs221B. In cases where the peptide backbone was longer than the new peptide sequence, all combinations of N-terminal and C-terminal truncations of the peptide backbone were tested. In cases where the length of the new peptide sequence exceeded that of the backbone, all possible combinations of N- and C-terminal extensions were tested by adding idealized helical residues. All subsequent design steps locked the peptide sequence. The hinge-peptide interface was designed in PyRosetta™ FastDesign™ for one repeat with fixed backbone followed by two repeats with flexible backbone, then sequences were improved by design with ProteinMPNN™ using a temperature of 0.2 and model version v_48_020. Structures of ProteinMPNN™ sequences were predicted with AlphaFold2™ using the ™design model as initial guess, and designs that predicted with mean pLDDT <92, RMSD to reference >1.5, or mean PAE interaction >5 were discarded. Poses were combined with state X models from the parent hinges, and residues were linked between states for PyRosetta™ multistate design with flexible backbone followed by MPNN multistate design with the same settings as previous ProteinMPNN™ design. An increased success rate was observed when performing ProteinMPNN™ multistate design with a 60%-40% bias toward state Y sequences. State Y structures were predicted with AF2-IG, and mean pLDDT, RMSD to reference, and mean PAE interaction cutoffs of 93, 1.5, and 5, respectively. State X structures were also predicted with AlphaFold2™ with the same cutoffs, excluding mean PAE interaction. 4 out of 9 possible pairs of parent hinge and peptide sequence produced AlphaFold2™-verified models. 20 designs were ordered, expressed, and tested for binding by fluorescence polarization (FP). A common failure mode for these designs was low levels of soluble expression, but 15/19 with sufficient expression for FP displayed detectable binding to the intended peptide. 9 were selected based on soluble expression levels and on-target affinity for further characterization. Only one design (CSW13, FIG. 10) was determined to have no off-target binding to the peptides in the parent set, while 4/9 designs still bound the original peptide with higher affinity than the intended one. The remaining 4 bound the intended peptide with the highest affinity, but also displayed binding to one or more other peptides; an example of this is shown in CSW20 (FIG. 10)

### Design of 3 Helix Bundles

Starting from AF2 models of validated hinge-peptide complexes, we sketched rough 3hb backbones in PyMOL™ by manually positioning two additional helices to buttress the bound helical effector peptide. For each sketch, we extracted the center four residues of the placed helices and used inpainting with RoseTTAFold™ to generate 1000 3hb backbones scaffolding those fragments onto the effector peptide. During the inpainting process, residues on the effector peptide interfacing with either of the placed helices were allowed to mutate; this and the placements of the four-residue fragments guided inpainting to build valid 3hb backbones that roughly aligned with the sketches. For the best 10% (by RoseTTAFold™ pLDDT) of backbones generated from each sketch, sequences were optimized using ProteinMPNN™. AF2-IG was used to predict the structure of the designed 3hbs with and without their target hinge, selecting only those designs which retained the same structure in both predictions and bound their target hinge with the same interface as the original effector peptide. We experimentally characterized the 1-3 designs per sketch that showed the best PAE interaction in the bound prediction, pLDDT in both predictions, and structural diversity (by eye).

### Design of Hinge-Armed Trimers

To fuse hinge cs221 to the asymmetric unit (asu) of a validated C3-symmetric homotrimer (35, 36), we manually positioned the two proteins such that they formed a large interface, their termini were near, and the angle of hinge switching was approximately perpendicular to the homotrimer axis of symmetry. We used inpainting with RoseTTAFold™ to generate 100 loop backbones between the N-terminus of cs221 and the homotrimer asu, allowing residues in the interface between the two proteins to mutate. To improve visibility of the conformational change in nsEM, we extended the C-terminal end of cs221 by fusing it to LHD101B, a previously validated monomeric protein (37). Again, we manually positioned the two proteins such that they formed a large interface and their termini were near, then used inpainting with RoseTTAFold™ to generate 100 loop backbones between those termini, allowing residues in the interface to mutate. For the best 20% (by RoseTTAFold™ pLDDT) of backbones generated for each fusion, we optimized sequences of the fusion region using ProteinMPNN™. We combined the most confidently-predicted (by AF2 pLDDT) LHD101B fusion with each homotrimer asu fusion, modeled each symmetric complex by aligning three copies of each fusion to the original homotrimer, and used AF2-IG to predict the symmetric structure of the designed fusions. We experimentally characterized the 7 most confidently-predicted designs.

### Hinge Extension for FRET Constructs

Hinges were extended by aligning a copy of the parent DHR to the first repeat of the hinge and another copy of the parent DHR to the last repeat of the hinge. The extended hinge was then obtained by replacing the first and last repat of the hinge by 2 or more repeats from the parent DHR. For cs221F, the additional repeats were redesigned using ProteinMPNN™

### Disulfide Stapling

A custom PyRosetta™ script was used to identify candidate positions for disulfides that could lock hinges in one conformation. i-j residue pairs where residue i is in domain 1 of the hinge and residue j is in domain 2 of the hinge were exhaustively evaluated using a 6D hashing protocol (51). For each candidate pair, 2 separate pdbs were generated for state X and state Y of the hinge with the identified residues i and j mutated to cysteine. AF2-IG was used to filter candidate pairs, selecting only pairs for which the cysteine side chains in the “target” state showed distances and relative orientations compatible with disulfide formations and for which the “off-target” state showed a large distance between cysteine side chains.

### ProteinMPNN™-Based Identification of Point Mutant Candidates

ProteinMPNN™ was used to generate 100 sequences optimized for state X and another 100 sequences optimized for the state Y-peptide complex. For each state, consensus sequences (38) were used to identify non-interface positions with distinct residue preferences that were different between both states. For mutations that AF2 predicted to not affect the global structure, individual protein variants carrying these mutations were experimentally tested using the FP peptide binding assay.

### Cloning, Expression, and Protein Purification

Genes encoding for proteins and peptides were either purchased as pre-cloned genes from IDT in pet29B expression vectors or purchased as e-blocks from IDT and cloned into custom target vectors using golden gate assembly (48). Hinges and 3-helix bundles usually carried a C-terminal SNAC™ tag (52) followed by a 6×His-tag (Hinge-GSHHWGSTHHHHHH (SEQ ID NO:48)); in some cases the SNAC™ tag was omitted (Hinge-GSHHHHHH (SEQ ID NO:46)). Peptides were expressed fused to superfolder green fluorescent protein (sfGFP) in either a sfGFP-(linker)-peptide-(linker)-6×His construct or sfGFP-GSGSENLYFQS (SEQ ID NO:47)-(linker)-peptide-(linker)-6×His construct. All proteins were expressed either in LEMO21 or NEB BL21(DE3) E. coli cells by autoinduction using TBII media (Mpbio) supplemented with 50×5052, 20 mM MgSO4 and trace metal mix and 50 mg/l Kanamycin. Expression cultures were grown at 37° C. for 20-24 h or at 37° C. for 5-6 h followed by 24 h at 18° C.

After harvesting with centrifugation, cells were lysed at 4° C. with sonication in lysis buffer containing (100 mM Tris HCl pH 8, 200 mM NaCl, 50 mM imidazole, 1 mM PMSF, 1 mM DNase, 1 Pierce™ Protease Inhibitor Mini Tablets, EDTA-free per 100 mL) and clarified with ultracentrifugation at 14-20k×g for 20-40 min. The constructs were bound to −1 mL Ni-NTA resin (Qiagen) and mixed for 10-60 min. The beads were sequentially washed with 15 mL low salt wash buffer (20 mM Tris HCl pH 8, 200 mM NaCl, 50 mM imidazole), 15 mL high salt wash buffer (20 mM Tris HCl pH 8, 1 M NaCl, 50 mM imidazole), and 15 mL low salt wash buffer. Lysates and buffer were flowed over the resin either using gravity or a vacuum manifold. Proteins were eluted in 1.4 mL of elution buffer (20 mM Tris HCl pH 8, 200 mM NaCl, 500 mM imidazole), after a 0.4 mL pre-elution. In constructs with designed disulfides, copper phenanthroline was then added to the elution at a final concentration of 10 mM, and the resulting mixture was incubated overnight to encourage full formation of the disulfides. In all cases elutions were further purified by SEC/FPLC on Superdex™ 75 Increase 10/300 GL or Superdex™ 200 Increase 10/300 GL columns in TBS (20 mM Tris pH 8, 100 mM NaCl), with 0.5 or 1 mL fractionation between 8 and 20 mL. LC-MS was used to confirm correct molecular weight of all purified proteins.

### Protein Purification for Crystallography

Constructs were transformed into LEMO21 or NEB BL21(DE3) E. coli and then expressed as 0.5 L cultures in 2L flasks. Proteins were expressed in Studiers M2 autoinduction media with 50 ug/mL kanamycin. Pre-cultures were grown at 37° C. for 4 hrs, then 22° C. for 14 hr and cultures were inoculated with 10 mL of preculture. Cells were pelleted at 4,000 g for 10 minutes, after which the supernatant was discarded. Pellets were resuspended in 40 mL of lysis buffer (100 mM Tris HCl pH 8, 100 mM NaCl, 400 mM imidazole, 1 mM PMSF, 1 mM DNase). Cell suspensions were lysed by microfluidization on a Microfluidics M-100P at 18,000 psi, and the lysate was clarified at 14,000 g for 30 minutes. The His-tagged proteins were bound to 8 mL Ni-NTA resin (Qiagen) during gravity flow and washed with 10 mL lysis buffer and 30 mL high salt wash buffer (25 mM Tris HCl pH 8, 1 M NaCl, 40 mM imidazole), then 10mL SNAC™ cleavage buffer (100 mM CHES, 100 mM Acetone oxime, 100 mM NaCl, 500 mM GnCl, pH 8.6). (52) 40 mL SNAC™ cleavage buffer and 80 uL 1M NiCl2 was added and columns were closed and shook on a nutator for 12 hours in order to cleave. After cleavage the flowthrough was collected and concentrated prior to further purification by SEC/FPLC on a HiLoad 20/600 Superdex™ 75 pg column in TBS (20 mM Tris pH 8.0, 100 mM NaCl), with 14 mL fractionation between 100 and 290 mL.

### Peptide Synthesis

Peptides were synthesized in-house on a CEM Liberty Blue™ microwave synthesizer. All amino acids were purchased from P3 Biosystems. Oxyma Pure™ was purchased from CEM, DIC was purchased from Oakwood Chemical, diisopropyl ethylamine (DIEA) and piperidine were purchased from Sigma-Aldrich. Dimethylformamide (DMF) was purchased from Fisher Scientific and treated with an Aldraamine trapping pack prior to use. 5(6)-carboxytetramethylrhodamine carboxylic acid (5(6)-TAMRA) was purchased from Novabiochem. Synthesis was done on a 0.1 mmol scale on CEM Cl-MPA resin. Five equivalents of each amino acid were activated using 0.1 M Oxyma™ with 2% (v/v) DIEA in DMF, 15.4% (v/v) DIC, and coupled twice on resin for 2 min per coupling with microwave irradiation. For TAMRA labeled peptides, peptides were washed with DMF post-synthesis, then incubated for 3 h with 5(6)-TAMRA carboxylic acid (3 eq.), HATU (3 eq.), and DIEA (5 eq.) in DMF, then washed with DMF (3×) followed by DCM (3×) to prepare for global deprotection. Global deprotection was accomplished with a TFA/water/TIPS/2,2′-(ethylenedioxy)diethanethiol (92.5:2.5:2.5:2.5) mixture for 3 hours. This deprotection mixture was concentrated in vacuo to 2-3 mL, then precipitated in 30 mL of ice-cold ethyl ether, centrifuged, and decanted, then washed twice more with fresh ether and dried under nitrogen to yield crude peptide for high pressure liquid chromatography (HPLC) purification. The crude peptide was dried and dissolved in a minimal amount of ACN and water to where the entire crude is soluble. This solution was purified on a Zorbax Stablebond™ C18 (9.4×250 mm, Sum) column using an Agilent 1260 Infinity™ HPLC. A linear gradient of water (0.1% TFA) and increasing ACN (0.1% TFA) was used to purify the crude peptides. UV signal was monitored at 214 nm and all peaks were collected. Peak masses were checked using an Agilent G6230B LC-MS and purity was assessed using a C18 column (Higgins Analytical PROTO 300 C18, 10 um, 10×250 mm) on an analytical Agilent 1260 Infinity™ II HPLC.

### SEC Binding Assay

Individual hinge and sfGFP-fused peptides or 3hb were diluted in 20 mM Tris pH 8, 100 mM NaCl and mixed at approximately 1:1 concentrations. 0.5-1 mL of the resulting samples were injected onto a Superdex™ 200 Increase 10/300 GL columns and the absorbance at 230 nm was used as a readout for binding. For sfGFP-fused peptides, 473 nm was also used as readout. Mixtures were at a total concentration of 2. μM or higher.

### Fluorescence Polarization (FP)

All FP measurements were performed at 25° C. in 96-well plates (Corning 3686) using a Synergy Neo2™ plate reader and a 530/590 nm filter cube. The buffer for all FP measurements was 20 mM Tris-HCl, 100 mM NaCl, 0.05% v/v TWEEN20 at pH 8. Titrations were carried out in 96-well format, with 4 replicates per plate and 24 data points per titration (23 steps of two-fold serial dilution of hinges in the presence of TAMRA-labeled peptide at a constant concentration between 0.1 nM and 1 nM) with a final sample volume of 80 μl per well. Titration plates were incubated overnight at room temperature before measuring to ensure complete equilibration. The polarization signal S (as calculated by the Neo2™ software) was fitted to the equation

$$S = {S}_{0} + {S}_{1} * {f}_{A B}$$

$${f}_{A B} = \frac{1}{2 {B}_{tot}} ( {A}_{tot} + {B}_{tot} + {K}_{D} - \sqrt{{( {A}_{tot} + {B}_{tot} + {K}_{D} )}^{2} - 4 * {A}_{tot} * {B}_{tot}} )$$

where f_{AB} is the fraction of peptide that is bound, Aₜₒₜ is the absolute hinge concentration, Bₜₒₜ is the absolute peptide concentration, S₀ is the baseline polarization of free peptide, and S₁ is the change in polarization upon complex formation.

Fitting was performed using the scipy.optimize.curve_fit python function (53). Uncertainties for K_{D} values are standard deviation errors calculated from the covariance matrix of the fits. In cases where the fitted K_{D} was lower than the concentration of the labeled peptide Bₜₒₜ, we report the K_{D} as K_{D}<Bₜₒₜ.

For FP kinetics experiments a 2× peptide solution and 8 different 2× hinge solutions at different concentrations were prepared separately. 40 μl of each hinge solution were mixed with 40 μl peptide solution using a multichannel pipet and the measurement was started immediately after mixing. Polarization signals S at each concentration were fitted individually to the equation

$$S = {S}_{0} - {S}_{1} * {e}^{- {k}_{app} ( {t}_{0} + t )}$$

where S₀ is the amplitude, S₁ is the polarization at equilibrium, kₐₚₚ is the apparent rate constant, t is the time after start of the measurement, and t₀ is the dead time between mixing and start of the measurement. For each hinge-peptide pair, the apparent rate constants for 8 different concentrations are fitted to the equation

$${k}_{app} = {k}_{off} + {k}_{o n} * {A}_{tot}$$

where k_{off} and kₒₙ are observed off- and on rates and Aₜₒₜ is the absolute hinge concentration.

### FRET

AlexaFluor™ 555 C2 maleimide (donor) and AlexaFluor™ 647 C2 maleimide (acceptor) were purchased from ThermoFisherScientific. Stock solutions at −5 mM were prepared by dissolving 1 mg of each dye in 200 μl DMSO. Hinge variants containing two cysteines were expressed and purified as described above with the modification that 0.5 mM TCEP was used during lysis, IMAC and SEC, and that the buffer for initial SEC contained 20 mM sodium phosphate (PH 7.0) instead of Tris-HCl. After SEC, 500 μl hinge at a concentration of 50 μM was incubated with 500 μM of a single dye for controls or 250 μM each of two dyes. After 2 h incubation at room temperature, samples were purified by SEC using a buffer containing 20 mM Tris-HCl and 100 mM NaCl at pH 8. LC-MS showed no residual unlabeled protein, suggesting complete labeling. UV-Vis analysis showed donor/acceptor ratios around between 40:60 and 60:40 for all double-labeled proteins.

The buffer for all FRET measurements was 20 mM Tris-HCl, 100 mM NaCl, 0.05% v/v TWEEN®20 at pH 8. Fluorescence spectra were recorded at room temperature using a FluoroMax™ spectrometer in a 1 cm×1 cm cuvette at a sample volume of 3 ml. FRET titrations and kinetics measurements were performed at 25° C. in 96-well plates (Corning 3686) using a Synergy Neo2™ plate reader. Excitation wavelength was 520 nm and emission wavelength was 665 nm (except for donor-donor controls for which emission wavelength was 555 nm, see FIG. 13B right column).

Titrations were carried out in 96-well format, with 4 replicates per plate and 24 data points per titration (23 steps of two-fold serial dilution of effector (peptide or 3hb) in the presence of double-labeled hinge at a constant concentration of 2 nM) with a final sample volume of 80 μl per well. Titration plates were incubated overnight at room temperature before measuring to ensure complete equilibration. The raw fluorescence signal (donor emission upon acceptor excitation) was fitted to the equation

$$S = {S}_{0} + sign ⋆ {S}_{1} * {f}_{A B}$$

$${f}_{A B} = \frac{1}{2 {A}_{tot}} ( {A}_{tot} + {B}_{tot} + {K}_{D} - \sqrt{{( {A}_{tot} + {B}_{tot} + {K}_{D} )}^{2} - 4 * {A}_{tot} * {B}_{tot}} )$$

where f_{AB} is the fraction of hinge that is bound, Aₜₒₜ is the absolute hinge concentration, Bₜₒₜ is the absolute peptide concentration, S₀ is the baseline fluorescence of free hinge, S₁ is the change in fluorescence upon complex formation, and sign=−1 for cs 201F (which shows a decrease in FRET upon binding) and sign=1 for cs074F and cs221F (which show an increase in FRET upon binding.

Fitting was performed using the scipy.optimize.curve_fit python function (53). Uncertainties for K_{D} values are standard deviation errors calculated from the covariance matrix of the fits. In cases where the fitted K_{D} was lower than the concentration of the labeled hinge Bₜₒₜ, we report the K_{D} as K_{D}<Bₜₒₜ.

For FRET kinetics experiments a 2× hinge solution and 8 different 2× effector solutions at different concentrations were prepared separately. 40 μl of each effector solution were mixed with 40 μl hinge solution using a multichannel pipet and the measurement was started immediately after mixing. Fluorescence signals S at each concentration were fitted individually to the equation

$$S = {S}_{0} - sign * {S}_{1} * {e}^{- {k}_{app} ( {t}_{0} + t )}$$

where S₀ is the amplitude, S₁ is the Fluorescence at equilibrium, kₐₚₚ is the apparent rate constant, t is the time after start of the measurement, and to is the dead time between mixing and start of the measurement, and sign=−1 for cs 201F (which shows a decrease in FRET upon binding) and sign=1 for cs074F and cs221F (which show an increase in FRET upon binding. For each hinge-peptide pair, the apparent rate constants for 8 different concentrations are fitted to the equation

$${k}_{app} = {k}_{off} + {k}_{o n} * {B}_{tot}$$

where k_{off} and kₒₙ are observed off- and on rates and Bₜₒₜ is the absolute peptide concentration.

Given a constant concentration of labeled hinge throughout a given FRET experiment (titration or kinetics experiment), and assuming a two-species system in which states X and Y exhibit different FRET efficiencies, our quantitative evaluation should be independent of labeling efficiencies and donor/acceptor stoichiometries, as these would only affect the amplitude which is accounted for by fitting parameters S₀ and S₁.

### DEER-Spin Label Modeling and Site Selection

All spin label modeling and distance distribution predictions were performed using chiLife™ (32) with the off-rotamer sampling method (54). Briefly, for each site, a spin label rotamer library (55) was superimposed on the site of interest. From the rotamer library, 5,000-10,000 new side chain conformations were sampled by randomly selecting a rotamer from the library and applying small random perturbations to the side chain dihedral angles (χ₁, χ₂, χ₃, χ₄, and χ₅ for the R1 spin label). Every rotamer sampled undergoes a clash evaluation using a modified Lennard-Jones potential and is reweighted based on this potential and the original weight of the parent rotamer in the rotamer library. Low weight (bottom 0.5%) rotamers are discarded and the weights of the remaining rotamers are normalized and summed to one. A more detailed description can be found in reference (54). To calculate a distance distribution between two rotamer ensembles, a weighted histogram is made for pairwise distances between the spin centers of each rotamer ensemble. The histogram is then convolved with a gaussian distribution with a 1 Å standard deviation. The resulting distribution is normalized such that the probability density sums to 1.

For each construct, spin label models were made for every site with at least 50 Å² solvent accessible surface area (SASA) in both conformational states. Pairwise distance distributions were predicted for all modeled spin labels in both states. Site pairs with the largest earth mover's distance (EMD) between the bound and unbound states were manually inspected and site pairs were selected that were predicted to have minimal interference with peptide binding, and conformational change. Two site pairs were chosen for each construct, one predicted to shift the distance distributions to a larger distance upon interaction with substrate and one predicted to shift to a shorter distance.

### DEER-Sample Preparation

Hinge variants carrying two cysteines were purified as described above but with 1 mM TCEP added to the lysis buffer and 0.5 mM TCEP added to an intermediate wash buffer. Directly after elution, 50 μL of 200 mM MTSL solution (in DMSO) was added to the entire 1.3 mL elution. After 1-6 h incubation at room temperature the labeling mixture was sterile filtered and purified by SEC. Successful labeling was confirmed by LC-MS.

Before DEER, 20 μM protein samples were prepared in 20 mM tris, 100 mM NaCl at pH 8.0 in D2O and 20% d8-glycerol (Cambridge Isotope Laboratories, Inc.) supplemented with 100 μM B-peptide when appropriate. Samples (20-40 L) were transferred to quartz capillaries (Sutter Instruments) with an inner diameter of 1.1 mm and an outer diameter of 1.5 mm, flash frozen with liquid nitrogen and stored at −80° C.

### DEER-Measurements

All DEER experiments were performed on an ELEXSYS® E580 EPR spectrometer (Bruker) at Q-band (˜34 GHz) using an EN5107D2 resonator (Bruker). A cryogen free cooling system (ColdEdge) was used to maintain a temperature of 50 K. Shaped pulses were generated using a SpinJet™ arbitrary waveform generator (Bruker). Observer pulses were 60 ns gaussian pulses with a full width at half maximum (FWHM) of 30 ns performed at approximately the center of the field-swept spectrum. Pump pulsers were 150 ns sech/tanh pulses centered 80 MHz above the observer pulses. Sech/tanh pulses were generated using PulseShape™ or EasySpin™ (56) with an excitation bandwidth of 80 MHz and a truncation parameter of 10. All sech/tanh pulses were modified to compensate for resonator performance and transmitter nonlinearity. All experiments used 8-step phase cycling and 8-step τ₁ averaging with 16 ns increments from 400 ns to 528 ns. Pump pulse time steps (Δt) and τ₂ times were chosen on a per sample basis and the values for each sample are reported in Table 6. Additional parameters including to offsets, shot repetition time, total number of averages, and more are reported in Table 6.

DeerLab (57) was used to analyze all DEER data to simultaneously fit foreground and background using Tikhonov regularization and compactness regularization (58). Akaike information criterion (AIC) and the information complexity criterion (ICC) were used to select regularization parameters for Tikhonov and compactness regularizations respectively. Sample fitting parameters including modulation depth, estimated signal to noise, smoothing and compactness regularization parameters are reported in Table 6.

### X-Ray Crystallography

All crystallization experiments were conducted using the sitting drop vapor diffusion method. Crystallization trials were set up in 200 nL drops using the 96-well plate format at 20° C. Crystallization plates were set up using a Mosquito™ from SPT Labtech, then imaged using UVEX microscopes and UVEX PS-600 from JAN Scientific. Diffraction quality crystals formed for 3hb05 in 0.2 M Lithium sulfate, 0.1 M Na-Phosphate-citrate pH 4.2, 20% PEG 1000; for 3hb12 1.8 M Ammonium citrate tribasic pH 7.0; for cs074AB in 0.2 M Calcium acetate, 0.1 M Na cacodylate pH 6.5, 40% PEG 300; for cs207A in 0.1 M SPG buffer pH 7, 25% (w/v) PEG 1500; for cs207AB in 0.2 M Magnesium sulfate and 20% (w/v) PEG 3350.

Diffraction data was collected at the Advanced Light Source beamlines 8.2.2/8.2.1. X-ray intensities and data reduction were evaluated and integrated using XDS (59) and merged/scaled using Pointless/Aimless™ in the CCP4 program suite (60). Structure determination and refinement starting phases were obtained by molecular replacement using Phaser™ (61) using the designed model for the structures. Following molecular replacement, the models were improved using phenix.autobuild (62); efforts were made to reduce model bias by setting rebuild-in-place to false, and using simulated annealing and prime-and-switch phasing. Structures were refined in Phenix™ (62). Model building was performed using COOT (63). The final model was evaluated using MolProbity™ (64). Data collection and refinement statistics are recorded in Table 7. Data deposition, atomic coordinates, and structure factors reported in this paper have been deposited in the Protein Data Bank (PDB), http://www.rcsb.org/with accession code 8FIH (3hb05), 8FVT (3hb12), 8FIT (cs074AB), 8FIN (cs207A) and 8FIQ (cs207AB).

### Negative Stain Electron Microscopy

Carbon-coated 400 mesh copper grids (01844-F, TedPella, Inc.) were first glow-discharged using a PELCO easiGlow™ cleaning System. SEC-purified proteins were diluted to 2 μg/ml with Tris Buffer (100 mM Tris, 40 mM NaCl), and then immediately pipetted onto the glow-discharged grid. The protein solution was allowed to sit on the grid for 30s, before being blotted away with Whatman filter paper. 3 uL of 2% uranyl formate stain was added to the grid and then blotted away after 10s. A second and third wash of UF stain was added to the grid, allowed to sit for 10s and 30s respectively, before being blotted away. The grid was allowed to air-dry for 5 minutes. Dried grids were then imaged using a FEI Talos™ L120C TEM (FEI Thermo Scientific, Hillsboro, OR) equipped with a 4K×4K Gatan OneView™ camera, at a magnification of 57,000× and pixel size of 2.49 Å. Once a grid-square with satisfactory stain thickness and contrast was identified, EPU software was used to automatically collect 200-400 micrographs across the square. Micrographs were imported into and analyzed using cryoSPARC™ v4.0.3. 50-100 particles were manually picked and subjected to 2D classification to find coarse 2D averages that could be used as templates for automated picking of thousands of particles across all micrographs. After automated picking and particle extraction from micrographs, a further round of 2D classification was done to find higher resolution averages of the hinge-bearing cyclic ring proteins in various states and orientations.

TABLE 5
Kinetic and thermodynamic parameters

| Hinge | Effector | K_{D} (nM) | K_{D} error (nM) | kₒₙ (M⁻¹ s⁻¹) | method |
|---|---|---|---|---|---|
| cs074 | TAMRA-cs074B | 0.5 | 0.9 | 64000 | FP |
| cs074F | cs074B | <2 | — | 170000 | FRET |
| cs074F | TAMRA-cs074B | <2 | — | 100000 | FP |
| cs074F | 3hb03 | <2 | — | — | FRET |
| cs074F | 3hb04 | <2 | — | — | FRET |
| cs074F | 3hb05 | <2 | — | — | FRET |
| cs074F | 3hb08 | <2 | — | — | FRET |
| cs074F | 3hb11 | <2 | — | — | FRET |
| cs074F | 3hb12 | <2 | — | — | FRET |
| cs074F | Peptide cs201B | 2500 | 120000 | | FRET |
| cs074F | Peptide cs221B | 130 | 50 | | FRET |
| cs094 | TAMRA-cs094B | 830 | 146 | 9400 | FP |
| cs201 | TAMRA-cs201B | 22 | 3.2 | 6700 | FP |
| cs201F | cs201B | 20 | 10 | 4500 | FRET |
| cs207 | TAMRA-cs207B | 820 | 432 | — | FP |
| cs217 | TAMRA-cs217B | 20000 | 18000 | — | FP |
| cs221 | TAMRA-cs221B | 2 | 0.6 | 3400 | FP |
| cs221_A114T | TAMRA-cs221B | 0.2 | 0.7 | 27700 | FP |
| cs221_A73E | TAMRA-cs221B | 4.9 | 1.2 | 1900 | FP |
| cs221_L66T | TAMRA-cs221B | 38.3 | 6.6 | 2700 | FP |
| cs221_lockedY | TAMRA-cs221B | 12 | 2 | 790000 | FP |
| cs221_V111L | TAMRA-cs221B | 0.3 | 0.7 | 24300 | FP |
| cs221_V111L_A111T | TAMRA-cs221B | 0.1 | 0.3 | 76200 | FP |
| cs221F | cs221B | 21 | 7 | 2600 | FRET |
| cs221F | TAMRA-cs221B | 23 | 9 | — | FP |
| cs221F | 3hb21 | 2.2 | 2 | 13000 | FRET |
| cs230 | TAMRA-cs230B | 2800 | 500 | — | FP |
| cs269 | TAMRA-cs269B | 83 | 8 | 2500 | FP |
| js007 | TAMRA-js007B | 5.7 | 1.2 | 78000 | FP |

TABLE 6
DEER experimental metadata and parameters

| | | | Δt | | t0 offset | τ2 | SRT | | | site |
|---|---|---|---|---|---|---|---|---|---|---|
| Sample | λ | Scans | (ns) | SNR | (ns) | (μs) | (ms) | α | β | pair |
| cs074_site_pair_1_Apo | 0.57 | 85 | 11 | 67 | 97.2 | 3 | 1.53 | 3.1 | 0.2 | K36R1 |
| | | | | | | | | | | Q211R1 |
| cs074_site_pair_1_Holo | 0.45 | 50 | 11 | 50 | 82.8 | 3 | 1.53 | 0.91 | 0.51 | K36R1 |
| | | | | | | | | | | Q211R1 |
| cs074_site_pair_2_Apo | 0.44 | 31 | 16 | 45 | 73.6 | 5 | 2.04 | 2.5 | 8.6 | E19R1 |
| | | | | | | | | | | E179R1 |
| cs074_site_pair_2_Holo | 0.4 | 39 | 16 | 79 | 99.2 | 5 | 2.04 | 1.03 | 9.15 | E19R1 |
| | | | | | | | | | | E179R1 |
| cs094_site_pair_1_Apo | 0.47 | 74 | 16 | 72 | 68.8 | 5 | 2.04 | 1.35 | 1.03 | R30R1 |
| | | | | | | | | | | E206R1 |
| cs094_site_pair_1_Holo | 0.4 | 83 | 16 | 43 | 80 | 5 | 2.04 | 1.7 | 2.98 | R30R1 |
| | | | | | | | | | | E206R1 |
| cs094_site_pair_2_Apo | 0.53 | 49 | 10 | 92 | 68 | 3 | 2.04 | 0.11 | 8.79 | K12R1 |
| | | | | | | | | | | E178R1 |
| cs094_site_pair_2_Holo | 0.55 | 69 | 16 | 72 | 67.2 | 5 | 2.04 | 0.1 | 1.36 | K12R1 |
| | | | | | | | | | | E178R1 |
| cs129_site_pair_1_Apo | 0.5 | 94 | 22 | 85 | 70.4 | 7 | 2.04 | 0.13 | 2.37 | E25R1 |
| | | | | | | | | | | K176R1 |
| cs129_site_pair_1_Holo | 0.45 | 6 | 12 | 50 | 54 | 4 | 2.04 | 0.62 | 1.13 | E25R1 |
| | | | | | | | | | | K176R1 |
| cs201_site_pair_1_Apo | 0.35 | 55 | 12 | 91 | 90 | 3.5 | 2.04 | 0.42 | 9.23 | K24R1 |
| | | | | | | | | | | E180R1 |
| cs201_site_pair_1_Holo | 0.36 | 90 | 12 | 86 | 76.8 | 3.5 | 2.04 | 0.18 | 2.93 | K24R1 |
| | | | | | | | | | | E180R1 |
| cs201_site_pair_2_Apo | 0.49 | 9 | 8 | 61 | 77.6 | 3 | 2.04 | 0.46 | 9.16 | R35R1 |
| | | | | | | | | | | E207R1 |
| cs201_site_pair_2_Holo | 0.5 | 83 | 8 | 154 | 76 | 3 | 2.04 | 0.14 | 5.42 | R35R1 |
| | | | | | | | | | | E207R1 |
| cs207_site_pair_1_Apo | 0.49 | 85 | 16 | 122 | 84.8 | 5 | 2.04 | 0.24 | 8.68 | D25R1 |
| | | | | | | | | | | K130R1 |
| cs207_site_pair_1_Holo | 0.5 | 69 | 16 | 99 | 59.2 | 5 | 2.04 | 0.15 | 0.24 | D25R1 |
| | | | | | | | | | | K130R1 |
| cs207_site_pair_2_Apo | 0.5 | 25 | 16 | 62 | 76.8 | 5 | 2.04 | 0.25 | 0.82 | K10R1 |
| | | | | | | | | | | D150R1 |
| cs207_site_pair_2_Holo | 0.46 | 25 | 8 | 56 | 86.4 | 5 | 2.04 | 0.4 | 1.32 | K10R1 |
| | | | | | | | | | | D150R1 |
| cs217_site_pair_1_Apo | 0.51 | 63 | 16 | 91 | 84.8 | 5 | 2.04 | 0.15 | 8.61 | R27R1 |
| | | | | | | | | | | E147R1 |
| cs217_site_pair_1_Holo | 0.49 | 74 | 16 | 57 | 72 | 5 | 2.04 | 0.48 | 8.91 | R27R1 |
| | | | | | | | | | | E147R1 |
| cs217_site_pair_2_Apo | 0.52 | 32 | 6 | 35 | 69.6 | 2 | 2.04 | 0.15 | 0.18 | E47R1 |
| | | | | | | | | | | R127R1 |
| cs217_site_pair_2_Holo | 0.54 | 17 | 10 | 37 | 47 | 3 | 2.04 | 0.13 | 6.17 | E47R1 |
| | | | | | | | | | | R127R1 |
| cs221_site_pair_1_Apo | 0.43 | 47 | 16 | 62 | 76.8 | 5 | 2.04 | 0.86 | 0.25 | R23R1 |
| | | | | | | | | | | E150R1 |
| cs221_site_pair_1_Holo | 0.41 | 48 | 16 | 39 | 76.8 | 5 | 2.04 | 1.5 | 9.21 | R23R1 |
| | | | | | | | | | | E150R1 |
| cs221_site_pair_2_Apo | 0.5 | 69 | 10 | 98 | 82 | 3.5 | 2.04 | 0.02 | 0.63 | E43R1 |
| | | | | | | | | | | K131R1 |
| cs221_site_pair_2_Holo | 0.52 | 72 | 16 | 74 | 88 | 5 | 2.04 | 0.12 | 0.24 | E43R1 |
| | | | | | | | | | | K131R1 |
| DHR82_site_pair_1_Apo | 0.5 | 238 | 16 | 66 | 81.6 | 5 | 2.04 | 0.6 | 8.8 | K36R1 |
| | | | | | | | | | | A211R1 |
| DHR82_site_pair_1_Holo | 0.49 | 92 | 16 | 43 | 86.4 | 5 | 2.04 | 0.37 | 0.46 | K36R1 |
| | | | | | | | | | | A211R1 |
| js007_site_pair_1_Apo | 0.4 | 40 | 16 | 34 | 78.4 | 5 | 2.04 | 1.51 | 9.24 | D60R1 |
| | | | | | | | | | | R190R1 |
| js007_site_pair_1_Holo | 0.43 | 43 | 16 | 74 | 97.6 | 5 | 2.04 | 0.45 | 0.38 | D60R1 |
| | | | | | | | | | | R190R1 |
| js007_site_pair_2_Apo | 0.45 | 31 | 16 | 40 | 96 | 5 | 2.04 | 1.28 | 6.63 | Q10R1 |
| | | | | | | | | | | Q219R1 |
| js007_site_pair_2_Holo | 0.48 | 34 | 16 | 31 | 81.6 | 5 | 2.04 | 2.77 | 1.54 | Q10R1 |
| | | | | | | | | | | Q219R1 |
| cs074 | 0.51 | 78 | 16 | 75 | 73.6 | 5 | 2.04 | 1.17 | 0.6 | K36R1 |
| 3hb03_site_pair_1_Holo | | | | | | | | | | Q211R1 |
| cs074 | 0.49 | 21 | 16 | 75 | 81.6 | 5 | 2.04 | 1.18 | 1.03 | K36R1 |
| 3hb04_site_pair_1_Holo | | | | | | | | | | Q211R1 |
| cs074 | 0.5 | 60 | 16 | 106 | 83.2 | 5 | 2.04 | 0.37 | 8.76 | K36R1 |
| 3hb05_site_pair_1_Holo | | | | | | | | | | Q211R1 |
| cs074 | 0.49 | 21 | 16 | 63 | 83.2 | 5 | 2.04 | 0.19 | 1.19 | K36R1 |
| 3hb08_site_pair_1_Holo | | | | | | | | | | Q211R1 |
| cs074 | 0.48 | 236 | 16 | 192 | 80 | 5 | 2.04 | 0.34 | 0 | K36R1 |
| 3hb11_site_pair_1_Holo | | | | | | | | | | Q211R1 |
| cs074 | 0.47 | 73 | 16 | 102 | 84.8 | 5 | 2.04 | 0.77 | 3.49 | K36R1 |
| 3hb12_site_pair_1_Holo | | | | | | | | | | Q211R1 |
| cs221- | 0.36 | 186 | 18 | 151 | 84.6 | 7 | 2.04 | 0.57 | 9.16 | R23R1 |
| mut_site_pair_1_Apo | | | | | | | | | | E150R1 |
| cs221- | 0.41 | 48 | 16 | 43 | 76.8 | 5 | 2.04 | 1.55 | 6.76 | R23R1 |
| mut_site_pair_1_Holo | | | | | | | | | | E150R1 |
| cs221- | 0.42 | 158 | 18 | 66 | 81 | 7 | 2.04 | 1.69 | 18.68 | E43R1 |
| mut_site_pair_2_Apo | | | | | | | | | | K131R1 |

λ—Modulation depth
Δt—Pump pulse time step
SRT—Shot repetition time
α—Smoothness regularization parameter
β—Compactness regularization parameter

TABLE 7
Crystallographic data collection and refinement

| | 3hb05 (8FIH) | 3hb12 (8FVT) | cs074AB (8FIT) | cs207A (8FIN) | cs207AB (8FIQ) |
|---|---|---|---|---|---|
| Data Collection | | | | | |
| Space group | P 21 21 2 | I 4 | P1 | P 21 21 21 | P 43 21 2 |
| Cell dimensions | | | | | |
| a, b, c (Å) | 66.01, 119.75, | 67.67, 67.67, | 44.11, 45.36, | 22.86, 81.73, | 71.88, 71.88, |
| | 38.80 | 82.37 | 61.80 | 154.94 | 127.64 |
| α, β, γ (°) | 90, 90, 90 | 90, 90, 90 | 109.27, 94.54, 104.09 | 90, 90, 90 | 90, 90, 90 |
| Resolution (Å) | 44.35-2.2 | 47.86-3.07 | 57.45-2.75 | 43.66-2.3 | 47.72-2.66 |
| | (2.3-2.2) | (3.86-3.07) | (2.9-2.75) | (2.39-2.3) | (2.8-2.66) |
| R_{merge} | 0.030 | 0.098 | 0.077 | 0.1154 | 0.1361 |
| | (1.161) | (0.415) | (0.428) | (0.5973) | (4.34) |
| Rₚᵢₘ | 0.030 | 0.035 | 0.064 | 0.03218 | 0.03788 |
| | (0.531) | (0.145) | (0.369) | (0.1575) | (1.226) |
| I/σ(I) | 9.89 | 17.39 | 7.58 | 15.19 | 11.84 |
| | (2.16) | (5.59) | (0.57) | (4.77) | (0.60) |
| CC_{1/2} | 0.999 | 1 | 0.986 | 1 | 1 |
| | (0.852) | (0.978) | (0.769) | (0.99) | (0.661) |
| Completeness | 95.27 | 99.60 | 89.39 | 99.85 | 97.86 |
| (%) | (72.82) | (99.48) | (90.08) | (99.93) | (89.38) |
| Redundancy | 1.4 | 8.8 | 2.2 | 13.7 | 13.9 |
| | (1.0) | (9.1) | (2.3) | (14.9) | (13.3) |
| Refinement | | | | | |
| Resolution (Å) | 44.35-2.2 | 47.86-3.07 | 57.45-2.75 | 43.66-2.3 | 47.72-2.66 |
| | (2.3-2.2) | (3.86-3.07) | (2.9-2.75) | (2.39-2.3) | (2.8-2.66) |
| No. reflections | 15519 | 30894 | 10934 | 13722 | 9939 |
| | (1653) | (15864) | (1563) | (1431) | (1246) |
| R_{work}/R_{free} | 0.1908 | 0.2248 | 0.2457 | 0.2403 | 0.2691 |
| | (0.1892)/ | (0.2998)/ | (0.2901)/ | (0.2360)/ | (0.4680)/ |
| | 0.2478 | 0.2728 | 0.2984 | 0.2712 | 0.3067 |
| | (0.2495) | (0.3158) | (0.3345) | (0.3046) | (0.4463) |
| No. atoms | | | | | |
| Protein | 2490 | 1518 | 3871 | 2659 | 1538 |
| Water | 81 | 0 | 4 | 24 | 0 |
| Ligand | 10 | 0 | 0 | 0 | 0 |
| Ramachandran | 98.70/1.30/0.00 | 96.81/3.19/0.00 | 97.49/1.88/0.00 | 99.42/0.58/0/00 | 99.48/0.52/0.00 |
| Favored/allowed | | | | | |
| Outlier (%) | | | | | |
| R.m.s. deviations | | | | | |
| Bond lengths (Å) | 0.006 | 0.004 | 0.002 | 0.001 | 0.002 |
| Bond angles (°) | 0.560 | 0.590 | 0.440 | 0.300 | 0.380 |
| B_{factors} (Å²) | | | | | |
| Protein | 41.25 | 85.94 | 86.88 | 48.07 | 108.65 |
| Water | 38.54 | n/a | 75.76 | 44.70 | n/a |
| Ligand | 64.46 | n/a | n/a | n/a | n/a |

TABLE 8
Alignments of crystal structures to design models

| | 3hb05 | 3hb12 | cs074AB | cs207A | cs207AB |
|---|---|---|---|---|---|
| | (8FIH) | (8FVT) | (8FIT) | (8FIN) | (8FIQ) |
| Heavy atom | 1.48 | 1.83 | 1.73 | 1.62 | 1.72 |
| RMSD (Å) | | | | | |
| C_alpha | 0.46 | 0.87 | 1.07 | 0.83 | 0.71 |
| RMSD (Å) | | | | | |
| TM score | 0.97 | 0.91 | 0.95 | 0.94 | 0.95 |

We claim:

1. A polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1-20,
wherein (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted.

2. The polypeptide of claim 1, wherein the polypeptide comprises the amino acid sequence selected from SEQ ID NO:1-20.

3. The polypeptide of claim 1, wherein the polypeptide comprises the amino acid sequence selected from SEQ ID NO:1-3.

4. A fusion protein, comprising:
(a) the polypeptide of claim 1; and
(b) one or more functional domains fused to the N-terminus and/or C-terminus of the polypeptide.

5. The fusion protein of claim 4, wherein the one or more functional domains are selected from the group consisting of a detectable protein, a polypeptide binding domain for a target, a protein enzyme, and an oligomerization domain.

6. The fusion protein of claim 4, wherein the one or more functional domains comprise the amino acid sequence selected from SEQ ID NO:21-28, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted.

7. The fusion protein of claim 4, wherein the fusion protein comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:29-32, wherein any N-terminal M residue is optional and may be present or may be deleted, and any C-terminal GSGSHHWGSTHHHHHH (SEQ ID NO:44) or GSHHHHHH (SEQ ID NO:46) sequence is optional and may be present or may be deleted.

8. A polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:33-43 and 49-63;
wherein (i) any N-terminal M residue is optional and may be present or may be deleted, and (ii) any C-terminal GSGSHHWGSTHHHHHH (SEQ ID NO:44) sequence is optional and may be present or may be deleted.

9. A polypeptide comprising the amino acid sequence selected from SEQ ID NO:33-43 and 49-63; wherein (i) any N-terminal M residue is optional and may be present or may be deleted, and (ii) any C-terminal GSGSHHWGSTHHHHHH (SEQ ID NO:44) sequence is optional and may be present or may be deleted.

10. A nucleic acid encoding the polypeptide of claim 1, or encoding a polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:33-43 and 49-63, wherein (i) any N-terminal M residue is optional and may be present or may be deleted, and (ii) any C-terminal GSGSHHWGSTHHHHHH (SEQ ID NO:44) sequence is optional and may be present or may be deleted.

11. An expression vector, comprising the nucleic acid of claim 10 operatively linked to a promoter sequence.

12. A host cell comprising the expression vector of claim 11.

13. A kit, comprising:
(a) one or more polypeptide of claim 1; and
(b) one or more polypeptide comprising an amino acid sequence at least 75%, 80%, 85%, 90%, 91%, 92%, 93%, 94%, 95%, 96%, 97%, 98%, 99%, or 100% identical to the amino acid sequence selected from SEQ ID NO:33-43 and 49-63, wherein (i) any N-terminal M residue is optional and may be present or may be deleted, and (ii) any C-terminal GSGSHHWGSTHHHHHH sequence is optional and may be present or may be deleted.

14. The kit of claim 13, wherein the kit comprise on or more combinations selected from:
(a) a first polypeptide or fusion protein comprising the amino acid sequence of SEQ ID NO:1, 4, 18, or 29; and a second polypeptide comprising the amino acid sequence selected from the group consisting of SEQ ID NO:33-38, 49;
(b) a first polypeptide or fusion protein comprising the amino acid sequence of SEQ ID NO: 3, 10, 17, 19, 20, 31, or 32; and a second polypeptide comprising the amino acid sequence selected from the group consisting of SEQ ID NO:39 and 55;
(c) a first polypeptide or fusion protein comprising the amino acid sequence of SEQ ID NO:12; and a second polypeptide comprising the amino acid sequence selected from the group consisting of SEQ ID NO:40 and 56;
(d) a first polypeptide or fusion protein comprising the amino acid sequence of SEQ ID NO:14; and a second polypeptide comprising the amino acid sequence selected from the group consisting of SEQ ID NO:43 and 60;
(e) a first polypeptide or fusion protein comprising the amino acid sequence of SEQ ID NO:5; and a second polypeptide comprising the amino acid sequence of SEQ ID NO:50;
(f) a first polypeptide or fusion protein comprising the amino acid sequence of SEQ ID NO:6; and a second polypeptide comprising the amino acid sequence of SEQ ID NO:51;
(g) a first polypeptide or fusion protein comprising the amino acid sequence selected from SEQ ID NO:2, 7, and 30; and a second polypeptide comprising the amino acid sequence of SEQ ID NO:52;
(h) a first polypeptide or fusion protein comprising the amino acid sequence of SEQ ID NO:8; and a second polypeptide comprising the amino acid sequence of SEQ ID NO:53;
(i) a first polypeptide or fusion protein comprising the amino acid sequence of SEQ ID NO:9; and a second polypeptide comprising the amino acid sequence of SEQ ID NO:54;
(j) a first polypeptide or fusion protein comprising the amino acid sequence of SEQ ID NO:13; and a second polypeptide comprising the amino acid sequence of SEQ ID NO:59;
(k) a first polypeptide or fusion protein comprising the amino acid sequence of SEQ ID NO:14; and a second polypeptide comprising the amino acid sequence of SEQ ID NO:60;
(l) a first polypeptide or fusion protein comprising the amino acid sequence of SEQ ID NO:15; and a second polypeptide comprising the amino acid sequence of SEQ ID NO:61;
(m) a first polypeptide or fusion protein comprising the amino acid sequence of SEQ ID NO:16; and a second polypeptide comprising the amino acid sequence of SEQ ID NO:62; and
(n) a first polypeptide or fusion protein comprising the amino acid sequence of SEQ ID NO:11; and a second polypeptide comprising the amino acid sequence selected from the group consisting of SEQ ID NO:63;
wherein in the first polypeptide or fusion protein, (i) any N-terminal M residue is optional and may be present or may be deleted, (ii) any C-terminal GSGSHHWGSTHHHHHH (SEQ ID NO:44) sequence is optional and may be present or may be deleted, and (iii) any N-terminal HHHHHHSGGS (SEQ ID NO:45) sequence is optional and may be present or may be deleted.

15. A method for use of the polypeptide of claim 1, comprising any combination of steps as recited here.


SEQUENCE LISTING (63 sequences)

SEQ ID NO: 1 - protein, 123 aa, synthetic construct
NPDNEEAVKTAVRLARELLKVAEELKERAEKTGDPRLLLLAAEAIAWAIEAVFLAAKASENTEGALEAARAAVKLAEVAKRIAKLLQRDAKKEGDPELLKLALRALELAVRAVELAIKENPDN

SEQ ID NO: 2 - protein, 125 aa, synthetic construct
AIKRNPDNEEAIKTALRLARELRKVAKELIERARKTGDAELLKKALEAARVAVEAVRLAAEYNKENAEKMAELLVELAELAREVADVLIELAEKTGDPELLKKALEVLEEAVEAVRLAIEYDPDH

SEQ ID NO: 3 - protein, 88 aa, synthetic construct
SDDEEVKEVVKKALEAALKSKDEEVIRLLLLAAVLAAAAARSGSPEEKLEIAKKALELAMKSKDEEVIRLALLAAVLAARSDDEEVLK

SEQ ID NO: 4 - protein, 254 aa, synthetic construct
MSGDEEVQEAVERAEELREEAEELIKKARKTGDPELLRKALEALKEAVRAVKEAIKRNPDNEEAVKTAVRLARELLKVAEELKERAEKTGDPRLLLLAAEAIAWAIEAVFLAAKASENTEGALEAARAAVKLAEVAKRIAKLLQRDAKKEGDPELLKLALRALELAVRAVELAIKENPDNEEAVETAKRLAEELRKVAELLEERAKETGDPELQELAKRAKEVADRARELAKKSNPNNGSGSHHWGSTHHHHHH

SEQ ID NO: 5 - protein, 225 aa, synthetic construct
MSGDEEERLRQEVEKAEKELEKLAKQSTDEEVRKMVREVAKQLRRLAEEAIRQKDDEALRQATEVVKMVQEAVKVAQKTTDQKVILLLLAVALVAIKVAQLAIRSDDTEALRLARIVIRAVQLLVKLVQKTTDPEVRRTALRVAELLAKLAKEAIERNDEEALREASEVVKEVQELVKEAEKSTDEEEIRELLQRAEERIREAQERIREGSGSHHWGSTHHHHHH

SEQ ID NO: 6 - protein, 234 aa, synthetic construct
HHHHHHSGGSSDEEEARELIERAKEAAERAQEAAERTGDPRVRELARELKRLAQEAAEEVKRDPSSSDVNKALKLIVELIEQAVRILQAAEEVGDPELRELARELVRLLVEMAEQVQRNPSSEDEVRALKLVVQAIEAAIRAAHAAQRTGDPEVDKLAKELVRLAVEAAEEVMRNPSSEEVNEALKKIVKAIQEAVESLREAEESGDPEKREKARERVREAVERAEEVQRDPSS

SEQ ID NO: 7 - protein, 249 aa, synthetic construct
MSGDEEVQEAVERAEELREEAEELIKKARKTGDPELLRKALEALKEAVRAVREAIKRNPDNEEAIKTALRLARELRKVAKELIERARKTGDAELLKKALEAARVAVEAVRLAAEYNKENAEKMAELLVELAELAREVADVLIELAEKTGDPELLKKALEVLEEAVEAVRLAIEYDPDHEEAVETAKRLAEELRKVAELLEERAKETGDPELQELAKRAKEVADRARELAKKSNGSGSHHWGSTHHHHHH

SEQ ID NO: 8 - protein, 195 aa, synthetic construct
MSGTEDERRELEKVARKAIEAAREGNTDEVREQLQRALEIARESGSEEAFKLALEVVRRVAEVAARAGNVEAVKEALRVALEIVKEAMELIKDPEAIVRLALEAVRVVAEVAARAGAVEAVKVALRVALEIAKIAGTEEAVRLALEVVKRVSDIAKKAGNEDAVKEAEEVRKKIEEESGGSGSHHWGSTHHHHHH

SEQ ID NO: 9 - protein, 173 aa, synthetic construct
MSGEMKEEIRRLAEELEKKTKDEEVKELARKAAELAEKSDNEEVLEVVKEALRAALKSKDEEVRRLLLEAAVLAAAAESSGSPEEKLEVALRAIRLAEKSKDEEVRRLALEAAVLAAESDDEEVLEEVLRALRRAEESKDEEERREELREAVRRAREGSGSHHWGSTHHHHHH

SEQ ID NO: 10 - protein, 173 aa, synthetic construct
MSGEMKEEIRRLAEELRERTKDEEVRELAREAARLAEESDDEEVKEVVKKALEAALKSKDEEVIRLLLLAAVLAAAAARSGSPEEKLEIAKKALELAMKSKDEEVIRLALLAAVLAARSDDEEVLKKVKEALEKAMESKDVEEIRERLREAVEVARAGSGSHHWGSTHHHHHH

SEQ ID NO: 11 - protein, 239 aa, synthetic construct
MSGSEEVNERVKQLAEKAKEATDKEEVIEIVKELAELAKQSTDPNLVAEVVRALTEVAKTSTDTELIQEIVKVLLELARRLTDPQLLLEVLKSIAELLKELAEKTGNETAKLAALVAQIAAEVVEMALRAEKTHPGSEIVKLAVELVQKVAEIVLIAAQLMLDKPNSDEVRKVLKEVEKVAREALKALREAKRHPDSQKARDEIKEASQKAEEVKERIERAQEGSGSHHWGSTHHHHHH

SEQ ID NO: 12 - protein, 175 aa, synthetic construct
MSGTKEEKERIERIEKEVRSPDPENILEAVRKALELFRENPSEEAKELLKRAIEAALRSEDERAILEALRAALEAYRQGAGDREGLLEAIRVAIRRAAESEDERAILEAVRAARILLEAERTEEAKELLRVAIERAKKSPDPEAQREAKRAEEELRKEDGSGSHHWGSTHHHHHH

SEQ ID NO: 13 - protein, 184 aa, synthetic construct
MSGKEKAIKKLEEAIKLAEKVGSEEAKILKEIAEALIHAVKGDAEEAEEKARRASEEAKKVGSELAKILKLLAEALIHAVLAQKGDADAKEALKKAAELLKKAAEKVGSEEAKILKLIAEAVIAAVEGDKEKAIELLERAVELAKKVGSEEAKILKEIAEALIEAVKGGSGSHHWGSTHHHHHH

SEQ ID NO: 14 - protein, 194 aa, synthetic construct
MSGDEERVRELAERASREAERAVRSGSEESLEEALRNVEELIRKSDSEEARVRVAEAAVRAAAEAAARSGTVEAAAVAVIFTARVLQIVSSSEEAVVRVLAAAAAAAARVAVELGTEEALETALRFVELLVRLADSDEARQRVLRSASESARRSAEESGSERAREVAERFRERIERNQGSGSHHWGSTHHHHHH

SEQ ID NO: 15 - protein, 243 aa, synthetic construct
MSGDEELRQLLREAEELVREAREELKENPDDRETAEKAVRKAREALDRAREALKELPTEEAIRRLARIAKEAVEVAKRAVEAVPESERVLLEAARVALEAVEAARRALELAKGENTEEIEEAARILARVAKRAVEVAKRALEARPDSPELAEEAVKLAEEALEAAERALKVLPTDEAIEILREIAERAKEIARIARESVPDSPEVKKNAQRVEEKADRSLEEAEKRNGSGSHHWGSTHHHHHH

SEQ ID NO: 16 - protein, 253 aa, synthetic construct
MSGEEEERLRRAVEEAVREAERVREEAKKSGSEEAREVAREVREAAERAREVAEEAIRSGDPRALRLAEEVLEAVRKAAEVAVEAIKSGSEEAIELAREVAEAARRAAEVAREAIRVDDPRALRIAELAAEAVEAVAKAVEAAVLAIKSGSEEAIEAARLVAEAAYRAIEVAREAIRTGDERALELAREVLRSVIEAAKIAKEAIKSGSEEEIEKAIRVAKEAYREAERAERRIREGGSGSHHWGSTHHHHHH

SEQ ID NO: 17 - protein, 249 aa, synthetic construct
MSGNEEVKKLVEEAKKYLKEAEEYIKKAKKTGNEELLEKALKLLEKALELVKKAIELDPNNKEAIEQAVEIARALNEVAELLIEEAKKTGNEELLEKALEALEAANEALKLAIENNENDKELLAEIAVELAEVGVLLAEVLIEEAKKTGDPELLKLALEVLEAAVEALELALKLDPNNEEAVELAEEAAKLLEEVAKELKEEAKKTGDPELEKLAKEAEELAKRAKELAERQRGSGSHHWGSTHHHHHH

SEQ ID NO: 18 - protein, 249 aa, synthetic construct
MSGMEEVKELVEKAEELAKEAEELIKKAKKTGNPELLKEALRKLKEAVELAKEAVELDPEHEEAIELLARLAEKLLEVAKLLIELAKKTGNPELLKEALRALKEAREALRLAAEHAPEDAKLLARLAARLAELALLIAKILIELAKKTGNPELLLKALKVLTLAVEAVEAALEADPRLKEAVRVAKELAEVLKELAKLAKELAKKTGNEELKEAAKEAEEVAKRAEELAREAEGSGSHHWGSTHHHHHH

SEQ ID NO: 19 - protein, 173 aa, synthetic construct
MSGEMKEEIRRLAEELRERTKDEEVRELAREAARLAEESDDEEVKEVVKKALEAALKSKDEEVIRLLLLAAVCAAAAARSGSPEEKLEIAKKALELAMKSKDEEVIRLALLAAVLACRSDDEEVLKKVKEALEKAMESKDVEEIRERLREAVEVARAGSGSHHWGSTHHHHHH

SEQ ID NO: 20 - protein, 173 aa, synthetic construct
MSGEMKEEIRRLAEELRERTKDEEVRELAREAARLAEESDDEEVKEVVKKALEAALKSKDEEVIRLLLCAAVLAAAAARSGSPEEKLEIAKKALELAMKSKDEEVIRLACLAAVLAARSDDEEVLKKVKEALEKAMESKDVEEIRERLREAVEVARAGSGSHHWGSTHHHHHH

SEQ ID NO: 21 - protein, 112 aa, synthetic construct
DEEVQEAVERAEELREEAEELIKKARKTGDPELLRKALEALECAVRAVEEAIKRNPDNDEAVETAVRLARELKKVAEELQERAKKTGDPELLKLALRALKVAVRAVKLAIKS

SEQ ID NO: 22 - protein, 124 aa, synthetic construct
DEAVETAVRLARELKKVAEELQERAKKTGDPELLKLALRALEVAVRAVELAIKSNPDNEEAVETAKRLAEELRKVAELLEERACETGDPELQELAKRAKEVADRARELAKKSNPNNGSHHHHHH

SEQ ID NO: 23 - protein, 169 aa, synthetic construct
MSGDEEVQEAVERAEELREEAEELIKKARKTGDPELLRKALEALEEAVRAVEEAICRNPDNDEAVETAVRLARELKKVAEELQERAKKTGDPELLKLALRALEVAVRAVELAIKSNPDNDEAVETAVRLARELKKVAEELQERAKKTGDPELLKLALRALEVAVRAVEL

SEQ ID NO: 24 - protein, 132 aa, synthetic construct
DEAVETAVRLARELKKVAEELQERAKKTGDPELLKLALRALEVAVRAVELAIKSNPDNEEAVETAKRLAEELRKVAELLEERAKETGDPCLQELAKRAKEVADRARELAKKSNPNNGSGSHHWGSTHHHHHH

SEQ ID NO: 25 - protein, 76 aa, synthetic construct
MSGEMVEEIEKLAEELAKKTKDCEVKKLAKKAAELAKKSTDEMKKEIVKDALELALRTKDEEVIRLALKAAVLAAE

SEQ ID NO: 26 - protein, 87 aa, synthetic construct
IVKKALELAMESKDVEVIRLALEAAVLAARSTDPEKKKCVYEALEKAMESKDEEEIKKELKAAVEKARSTEGSGSHHWGSTHHHHHH

SEQ ID NO: 27 - protein, 210 aa, synthetic construct
MSGPELVLKALENMVRAAHTLAEIARDNGNEEWLEAAARLAELVAKAAEELAREARKEGNLELALKALQILVNAAYVLAEIARDRGNEELLEYAARLAEEAARQAAEIAIKAARKGNLELALEALRILNEAARVLARIAHHRGNQELLKKAQHLTEASAAISKMLAAIAAATGGGEMIGEIAGLAAELIFRTDDKEVRELAARAAGLAAG

SEQ ID NO: 28 - protein, 124 aa, synthetic construct
LVKKALEKAMESKNVEAIRNMLKVAVEAARSGGGRQWAVVLSIAETAGKMGVTMEFHVSGNEVKVVIKGLHESQQEQLLEDVLRTAEKQGVRVRIRFKGDTVTIVVREGSGSHHWGSTHHHHHH

SEQ ID NO: 29 - protein, 359 aa, synthetic construct
DEEVQEAVERAEELREEAEELIKKARKTGDPELLRKALEALECAVRAVEEAIKRNPDNDEAVETAVRLARELKKVAEELQERAKKTGDPELLKLALRALKVAVRAVKLAIKSNPDNEEAVKTAVRLARELLKVAEELKERAEKTGDPRLLLLAAEAIAWAIEAVFLAAKASENTEGALEAARAAVKLAEVAKRIAKLLQRDAKKEGDPELLKLALRALELAVRAVELAIKENPDNDEAVETAVRLARELKKVAEELQERAKKTGDPELLKLALRALEVAVRAVELAIKSNPDNEEAVETAKRLAEELRKVAELLEERACETGDPELQELAKRAKEVADRARELAKKSNPNNGSHHHHHH

SEQ ID NO: 30 - protein, 426 aa, synthetic construct
MSGDEEVQEAVERAEELREEAEELIKKARKTGDPELLRKALEALEEAVRAVEEAICRNPDNDEAVETAVRLARELKKVAEELQERAKKTGDPELLKLALRALEVAVRAVELAIKSNPDNDEAVETAVRLARELKKVAEELQERAKKTGDPELLKLALRALEVAVRAVELAIKRNPDNEEAIKTALRLARELRKVAKELIERARKTGDAELLKKALEAARVAVEAVRLAAEYNKENAEKMAELLVELAELAREVADVLIELAEKTGDPELLKKALEVLEEAVEAVRLAIEYDPDHDEAVETAVRLARELKKVAEELQERAKKTGDPELLKLALRALEVAVRAVELAIKSNPDNEEAVETAKRLAEELRKVAELLEERAKETGDPCLQELAKRAKEVADRARELAKKSNPNNGSGSHHWGSTHHHHHH

SEQ ID NO: 31 - protein, 251 aa, synthetic construct
MSGEMVEEIEKLAEELAKKTKDCEVKKLAKKAAELAKKSTDEMKKEIVKDALELALRTKDEEVIRLALKAAVLAAESDDEEVKEVVKKALEAALKSKDEEVIRLLLLAAVLAAAAARSGSPEEKLEIAKKALELAMKSKDEEVIRLALLAAVLAARSDDEEVLKIVKKALELAMESKDVEVIRLALEAAVLAARSTDPEKKKCVYEALEKAMESKDEEEIKKELKAAVEKARSTEGSGSHHWGSTHHHHHH

SEQ ID NO: 32 - protein, 422 aa, synthetic construct
MSGPELVLKALENMVRAAHTLAEIARDNGNEEWLEAAARLAELVAKAAEELAREARKEGNLELALKALQILVNAAYVLAEIARDRGNEELLEYAARLAEEAARQAAEIAIKAARKGNLELALEALRILNEAARVLARIAHHRGNQELLKKAQHLTEASAAISKMLAAIAAATGGGEMIGEIAGLAAELIFRTDDKEVRELAARAAGLAAGSDDEEVKEVVKKALEAALKSKDEEVIRLLLLAAVLAAAAARSGSPEEKLEIAKKALELAMKSKDEEVIRLALLAAVLAARSDDEEVLKLVKKALEKAMESKNVEAIRNMLKVAVEAARSGGGRQWAVVLSIAETAGKMGVTMEFHVSGNEVKVVIKGLHESQQEQLLEDVLRTAEKQGVRVRIRFKGDTVTIVVREGSGSHHWGSTHHHHHH

SEQ ID NO: 33 - protein, 119 aa, synthetic construct
MSGMAAKVREEAERVREEARETRERAREAAERGEVSEETAAALLLEAAVLELKAVLLELEARRIYEEAGPEEREVAEEARRLAEEARRLAREARELAREAEEGGSGSHHWGSTHHHHHH

SEQ ID NO: 34 - protein, 123 aa, synthetic construct
MSGGEAEELREEAERARRRAEEARERAREALERARRGEASEEHAAALLAEAAVLELKAVLLTLEARRLEKELGGDERAREALEAAEEARRAAREARREAEEAFEAASGSGSHHWGSTHHHHHH

SEQ ID NO: 35 - protein, 123 aa, synthetic construct
MSGEEAERLRREAERNREKAEEQREKAKKAYEKAKKGEASEEHAAALLAEAAVLELKAVLLTLEARRLYKELGGDERAREALEAAERAREAAREAREVAEKAYDAASGSGSHHWGSTHHHHHH

SEQ ID NO: 36 - protein, 120 aa, synthetic construct
MSGGEERAREVREEAERTRREAEELRERAEEALESGEASEEHAAALLLEAAVLELKAVLLELEARRLREEHPTEEAREAYEAAERAREAAREALEAAREALEKSGSGSHHWGSTHHHHHH

SEQ ID NO: 37 - protein, 120 aa, synthetic construct
MSGGKAEEARRLAEKALEAARKALEYARKAKEALEEGKSEEVVAAYLLEAAVEDLKAVLLRLEARRLAEESDEEARRETERLLEEARRAREEARRLRREIEERKGSGSHHWGSTHHHHHH

SEQ ID NO: 38 - protein, 119 aa, synthetic construct
MSGGLEEEARELAEEAREVRRRAEELRRRAEEARETGEASEEHAAALLAEAAVLELKAVLLELEARRLLKESGGEVAREALELAREARREAREALEAAEEASEGSGSHHWGSTHHHHHH

SEQ ID NO: 39 - protein, 94 aa, synthetic construct
MSGSKEEAKKEFMELARKKAKEIEENPEKARELAEEALKELEKKYEELKKAGVPEKEALALYVIALARVLIAKLAAEEGSGSHHWGSTHHHHHH

SEQ ID NO: 40 - protein, 90 aa, synthetic construct
MSGMEERYERAERLGREVAELLRRGAPEEEIRRAIEELLRLLDSLPLPPAGSSETARLIALAALAAAAELALDLGSGSHHWGSTHHHHHH

SEQ ID NO: 41 - protein, 90 aa, synthetic construct
MSGMEEKKRRIRELVEEARRLVEEGAPLEEIEERVREFAEELREIAKEAGKNSELKLMVAAALLILAALIIEKRGSGSHHWGSTHHHHHH

SEQ ID NO: 42 - protein, 100 aa, synthetic construct
MSGVEEKIKEAEELARRMREALKKYEETGDEKYKEEIEKLSERLRELTREILEKGNVEEKQKAVLLTAIRALIEIYLRLLEKEKGSGSHHWGSTHHHHHH

SEQ ID NO: 43 - protein, 103 aa, synthetic construct
MSGEEEMKRRIRELEREALRRLDEAARTGSVEELDRVYREERRRFEELREELRREGLEGSEVDLLWLEALAVVEAAYIGARVVLELAGSGSHHWGSTHHHHHH

SEQ ID NO: 44 - protein, 16 aa, synthetic construct
GSGSHHWGSTHHHHHH

SEQ ID NO: 45 - protein, 10 aa, synthetic construct
HHHHHHSGGS

SEQ ID NO: 46 - protein, 8 aa, synthetic construct
GSHHHHHH

SEQ ID NO: 47 - protein, 11 aa, synthetic construct
GSGSENLYFQS

SEQ ID NO: 48 - protein, 14 aa, synthetic construct
GSHHWGSTHHHHHH

SEQ ID NO: 49 - protein, 28 aa, synthetic construct
SEEQEAARLLELAVEDLKLVLDALEKRR

SEQ ID NO: 50 - protein, 23 aa, synthetic construct
SEVEELVEEVEELIKLLEEMTKR

SEQ ID NO: 51 - protein, 27 aa, synthetic construct
DESRERLKEEVKRLIEQLQKKNKKQQK

SEQ ID NO: 52 - protein, 30 aa, synthetic construct
SILELAEEVAEEIKEAIRIAAKGIREGLKE

SEQ ID NO: 53 - protein, 28 aa, synthetic construct
MEEARKELTREMIEVLREIEEGFKERFE

SEQ ID NO: 54 - protein, 26 aa, synthetic construct
EERKKKLAREVVREARRLIERLAEEE

SEQ ID NO: 55 - protein, 26 aa, synthetic construct
EERKKELAKEVIETAKKLIEKLAKEE

SEQ ID NO: 56 - protein, 27 aa, synthetic construct
SSETAREIARVALEAAKWLYEDLKAEE

SEQ ID NO: 57 - protein, 27 aa, synthetic construct
SEEKLAEAARLLIEAAEIIKERGEKEG

SEQ ID NO: 58 - protein, 28 aa, synthetic construct
VEKLQREVLEEAERALREILERLEEKEK

SEQ ID NO: 59 - protein, 28 aa, synthetic construct
SATDKKLEDIALEMGGYLAVLMVDILKK

SEQ ID NO: 60 - protein, 28 aa, synthetic construct
SEELLRELEELAVDEARRIGAEVVRRLA

SEQ ID NO: 61 - protein, 28 aa, synthetic construct
EKEEITEELREVLREAERLAEEVLRILE

SEQ ID NO: 62 - protein, 28 aa, synthetic construct
EVIRLGFEVAERVEELLREAREIIEATA

SEQ ID NO: 63 - protein, 26 aa, synthetic construct
EEEEEVHREMQKAVRKALEAIKRILK